(19) United States
(12) Patent Application Publication
(10) Pub. No.: US 2026/0348918 A1
(43) Pub. Date: Nov. 5, 2026

(54) METHODS AND DEVICES FOR SIMULATION OF AGITATION STRESS ON LIQUIDS

(71) Applicant: Hoffmann-La Roche Inc., Little Falls, NJ (US)

(72) Inventors: Gernot Kurt BOIGER, Winterthur (CH); Florian ROSENTHAL, Luzern (CH)

(73) Assignee: Hoffmann-La Roche Inc., Little Falls, NJ (US)

(21) Appl. No.: 19/177,423

(22) PCT Filed: Oct. 12, 2023
(86) PCT No.: PCT/EP2023/078272

§ 371 Date: Apr. 11, 2025

(30) Foreign Application Priority Data
Oct. 14, 2022 (EP) 22201593.5

(51) Int. Cl. G01N 29/02; B01F 31/22; G01N 29/34; G01N 33/15

(52) U.S. Cl. CPC G01N 29/02; B01F 31/22; G01N 29/34; G01N 33/15

(57) ABSTRACT

The present invention relates to a method for simulation of quality impact of transportation on a liquid, and a two-dimensional shaker adapted to perform said method.

### FIELD OF THE INVENTION

The present invention relates to a method for simulation of quality impact of transportation on a liquid, and a two-dimensional shaker adapted to perform said method.

### BACKGROUND OF THE INVENTION

When developing a pharmaceutical product, there are many factors that need to be considered. Among the many factors that are to be evaluated, product stability and integrity are essential to ensure efficacy and safety of a product. Especially when biological and biopharmaceutical products are developed the stability is of paramount importance. The complex molecular make-up of these large molecules make them highly vulnerable to changes in their conformation and structure. External factors such as temperature change, pH alterations and surface adsorption can induce protein aggregation, protein particles and chemical modifications [Das, T. K.; Carroll, J. A. Biophysical and Biochemical Characterization of Peptide, Protein, and Bioconjugate Products. In Parenteral Medications; Nema, S., Dubois, L., Eds.; FL: CRC Press-Taylor & Francis Group, 2019; pp 219-248; Nowak, C.; K. Cheung, J.; M. Dellatore, S.; Katiyar, A.; Bhat, R.; Sun, J.; Ponniah, G.; Neill, A.; Mason, B.; Beck, A.; Liu, H. Forced Degradation of Recombinant Monoclonal Antibodies: A Practical Guide. MAbs 2017, 9 (8), 1217-1230]. Therefore, a lot of development work is dedicated to finding formulations for the biopharmaceuticals that are able to conserve their structure and thereby assure their efficacy and safety [Das, T. K. et al, 2020; DOI: 10.1016/j.xphs.2019.09.023; Wang, W. et al, 2012; DOI: 10.1016/j.ijpharm.2012.04.040].

Several steps within the production and distribution process of biopharmaceuticals are responsible for creating stress conditions that can affect the proteins' structure. One of these steps is the transportation of the drugs to the patients [Das, T. K. et al, 2020, DOI: 10.1016/j.xphs.2019.09.023]. To administer biopharmaceuticals parenterally, they need to be in liquid formulation, and during transportation, the movements of the liquid induce shear forces or interfacial effects that can cause the proteins to degrade/aggregate. So, pharmaceutical companies have to test the robustness against vibrations, temperature changes, liquid-air interface, shear stress and other types of stresses that could disrupt the product quality [Nowak, C.; K. Cheung, J.; M. Dellatore, S.; Katiyar, A.; Bhat, R.; Sun, J.; Ponniah, G.; Neill, A.; Mason, B.; Beck, A.; Liu, H. Forced Degradation of Recombinant Monoclonal Antibodies: A Practical Guide. MAbs 2017, 9 (8), 1217-1230; Koepf, E. et al, DOI: 10.1016/j.ijpharm.2017.12.043; Maa, Y. F.; Hsu, C. C., 1997, DOI: 10.1002/(SICI) 1097-0290 (19970620) 54: 6<503: AID-BIT1>3.0.CO; 2-N]. One of the ways to circumvent this issue is to use lyophilisation. Lyophilisation is the process of removing water and transforming the solution into a solid cake or powder to increase shelf life and ease transportation. However, this process is very labour intensive and requires reconstitution, hence “ready-to-use” formulations are used [Bjelošević, M.; Zvonar Pobirk et al, 2020, DOI: 10.1016/j.ijpharm.2020.119029; Bye, J. W. et al, 2014, DOI: 10.1007/s10529-013-1445-6]. In addition to this, demand for convenient auto-injectors and prefilled syringes has increased, where lyophilized formulations are not an option [Bye, J. W. et al, 2014, DOI: 10.1007/s10529-013-1445-6; Sassalos, T. M., Paulus, Y. M., 2019, DOI: 10.2147/OPTH.S169044]. A way of preventing aggregation of liquid formulated biopharmaceuticals is to add certain non-active ingredients (excipients) to the drug product. Examples of these excipients include: disaccharides and surfactants while, changing the pH and changing the protein concentration can also prevent protein aggregation [Narhi, L. O. et al, 2022, DOI: 10.1016/j.xphs.2022.01.011; Das, T. K. et al, 2021, DOI: 10.1016/j.xphs.2021.09.030].

The FDA and other health authorities require a shipping validation study, where biological drug product robustness and quality after transport is assessed [Nowak, C.; K. Cheung, J.; M. Dellatore, S.; Katiyar, A.; Bhat, R.; Sun, J.; Ponniah, G.; Neill, A.; Mason, B.; Beck, A.; Liu, H. Forced Degradation of Recombinant Monoclonal Antibodies: A Practical Guide. MAbs 2017, 9 (8), 1217-1230; Narhi, L. O. et al, 2022, DOI: 10.1016/j.xphs.2022.01.011]. To assess product robustness and quality after shipping, pharmaceutical companies are required to do either a Real Life Shipping (RLS) test where a transportation of the new drug is sent through the representative distribution route (usually requested by FDA). For submission of a new biologic drug product to other health authorities, e.g. EMA, companies are required to do a simulation of transportation, a so-called ASTM (American Society for Testing and Materials) vibration test (ASTM D4169). There are three transportation methods (Rail, Truck and Air) and there are three intensity levels for each method (I, II, III) covered by the ASTM method. Currently, the guidelines advise a programme where levels I, II and III are consecutively tested for a designated time period [ASTM International. ASTM Standard Practice for Performance Testing of Shipping Containers and Systems; 2016; https://www.astm.org/d4169-14.html; ASTM International. ASTM D4169 Truck Profile Update Rationale ASTM D4169 Truck Profile Update Rationale; 2016].

However, research has shown that the current methods of product quality assessment after transportation stress are not very representative and rather expensive [Borocz, P, 2019, DOI: 10.1002/pts.2434; Németh, Z. et al, 2021, DOI: 10.14513/actatechjaur.00603]. Firstly, the ASTM D4169 vibration tests only cover the vertical vibrations, even though when transporting a drug product, there are three dimensions of vibration. There is limited knowledge and data on the transportation stresses that biological drug products experience in real-life and if the addition of another dimension changes the stress to which the drug products are exposed.

In view of such circumstances as described above, it is an object of the invention to provide a two-dimensional shaker in order to more accurately replicate the agitation stresses on liquid pharmaceutical formulations during transportation, as two-dimensional vibration is closer to real life shipping. The devices and methods described herein can be used, inter alia, for evaluation of liquid formulation during development process, and also in order to satisfy the quality requirements as demanded by health authorities.

### BRIEF DESCRIPTION OF THE DRAWINGS

FIG. 1. Different wave formations can be seen between horizontal (X-axis), vertical (Z-axis) and combined (XZ-axis) vibration. Representative images of 6 ml vials filled with 3.2 ml are shown. Stills are taken from high speed video footage (200 fps).

FIG. 2. Particle comparison between four different antibodies in liquid formulation shaken in 3 different axes (X, Z and XZ). Each antibody formulation was filled in 6 ml vials and shaken for 30 minutes with the ASTM D4169-16 air level I profile. N=3 for the agitated antibody formulations, N=1/2 for the controls. A: Visual inspection of the vials, accompanied by pictures of a vial with many particles (left) and a non-agitated control vial (right). B: Results of sub-visible particle count by light obscuration. Amount of particles per ml in the size ranges 2-4 μm, 5-9 μm and 10-24 μm; EP=EP black/white box; SD=Seidnenader.

FIG. 3. Comparison between 3 different temperatures, 5, 23 and 30 °C. The CEA antibody formulation was filled in 6 ml vials and shaken in the XZ-axis for 30 minutes with the ASTM D4169-16 air level I profile. N=3 for the agitated antibody formulations, N=1/2 for the controls. A: Visual inspection of the vials. B: Results of sub-visible particle count by light obscuration. Amount of particles per ml in the size ranges 2-4 μm, 5-9 μm and 10-24 μm; EP=EP black/white box; SD=Seidnenader.

FIG. 4. Particle comparison between 6 ml and 20 ml vials with the same vertical filling height (3.2 ml and 6.4 ml respectively) shaken in two different vial orientations (horizontal and vertical). The CEA formulation was filled in either 6 ml vials or 20 ml vials and shaken for 30 minutes in the XZ-axis with the ASTM D4169-16 air level I profile. N=3 for the agitated antibody formulations, N=2 for the controls. A: Visual inspection of the vials. B: Results of sub-visible particle count by light obscuration. Amount of particles per ml in the size ranges 2-4 μm, 5-9 μm and 10-24 μm; EP=EP black/white box; SD=Seidnenader.

FIG. 5. Visual inspection surfactant study.

FIG. 6. Light obscuration sub-visible particle analysis comparison between XZ- and Z-axis agitation with different concentrations of PS80 at different time intervals. The CEA antibody formulations were filled in 6 ml vials and shaken in the XZ-axis (A) or Z-axis (B) for 15, 30, 60 or 120 minutes with the ASTM D4169-16 air level I profile. N=1 for all antibody formulations. A: sub-visible particle analysis by light obscuration: XZ vibration. B: sub-visible particle analysis by light obscuration: Z vibration.

FIG. 7. Background membrane imaging sub-visible particle analysis comparison between XZ- and Z-axis agitation with different concentrations of PS80 at different time intervals. The CEA antibody formulation was filled in 6 ml vials and shaken in the XZ-axis (A) or Z-axis (B) for 15, 30, 60 or 120 minutes with the ASTM D4169-16 air level I profile. N=1 for all antibody formulations, three samples were taken from one vial. BMI=Background Membrane Imaging. A: sub-visible particle analysis by BMI: XZ vibration. B: sub-visible particle analysis by BMI: Z vibration.

FIG. 8. Surface tension results from CEA with different concentrations of PS80.

FIG. 9. 6 mL vial in single parcel packaging configuration with double leaflet (left) for water-filled vials and single leaflet (right) for drug product vials.

FIG. 10. Schematic mass-spring system. A: spring; B: body with fixed motion; C: vibration amplification; D: rigid transmission; E: wave damping.

FIG. 11. Schematic path of the waves through packaging. A: Thermally insulating including cooling; B: packaging; C: secondary packaging; D: pallet; E: vial; F: path of the wave; G: surface of the vehicle (e.g. plane or truck).

FIG. 12. Location of Accelerometer measurements and reference sensors (left) MSR165: Logger with internal battery and sensor (right). A: Probe measurements; B: Reference measurements. The amplitude ration can be calculated by dividing Probe measurements by Reference measurement.

FIG. 13. Accelerometer within their holder are placed within a shipping box (exemplary).

FIG. 14. Temperature during transportation.

FIG. 15. Total vibration energy (Grms) during transportation, in the three axes X, Y and Z.

FIG. 16. PSD distribution and densities. A: Pallet in refrigerated Truck; Z axis data; B: Pallet in EKR1 Active Thermal shipper (truck); Z axis data; C: Pallet in EKR1 Active Thermal shipper (flight); Z axis data; D: Pallet in refrigerated truck; Z axis data; E: Pallet in refrigerated truck; Y axis data; F: Pallet in refrigerated truck; X axis data; G: Pallet in refrigerated truck; Z axis data. In PSD profiles of all the panels, the assurance levels (level I, II and III, as described in Example 2) are indicated by solid lines. For comparison, the relevant ASTM D4169-16 guideline profiles are plotted as dotted lines.

FIG. 17. Shocks recorded during transportation.

FIG. 18. Comparison of data from horizontal shaking during formulation development and real life shipment. Panel A: Sub-visible particles (SvP). Panel B: Size-exclusion high-performance liquid chromatography (SE-HMW). In both panels A and B the labels are: A: Active 2 formulation; B: Active 3 formulation; C: Active 1 formulation; D: Active 1 formulation, including 0.0.1% surfactant, after shaking for 7 days at 5 °C; E: Active 1 formulation, including 0.0.1% surfactant, after shaking for 7 days at 25 °C; F: Active 1 formulation, without surfactant, after shaking for 7 days at 5 °C; G: Active 1 formulation, without surfactant, after shaking for 7 days at 25 °C.

### DETAILED DESCRIPTION OF THE INVENTION

As used in the following, the terms “have”, “comprise” or “include” or any arbitrary grammatical variations thereof are used in a non-exclusive way. Thus, these terms can both refer to a situation in which, besides the feature introduced by these terms, no further features are present in the entity described in this context and to a situation in which one or more further features are present. As an example, the expressions “A has B”, “A comprises B” and “A includes B” can both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention can, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

Further, as used herein, transportation refers to movement of objects from one place to another. Modes of transport include air, land (rail and road), water, cable, pipeline, and space. In the context of this document, transportation refers particularly to the first three modes. The terms “transportation”, “transport”, “shipment” and “shipping” are used herein interchangeably and refer to the same concept.

In one aspect, provided herein is a method for simulation of quality impact of transportation on a liquid, said method comprising the steps of:
a) selecting a set of instructions comprising i) one or more PSD profile(s) comprising a multiplicity of frequencies and their corresponding amplitudes in at least two perpendicular axes (e.g. X and Z), and ii) a timetable specifying the duration for each of said PSD profile(s);
b) inducing vibrations in the liquid according to said PSD profile(s); and
c) quality assessment and comparison of said liquid before and after performing the step b.

PSD (Power Spectral Density or, as they are often called, acceleration spectral densities or ASD for vibration) plots are well known in the art and are used to quantify and compare different vibration environments. The power spectral density (PSD) of the wave (e.g. vibration) describes the power present in the wave as a function of frequency, per unit frequency. Power spectral density is commonly expressed in watts per hertz (W/Hz) or g²/Hz in which g denotes the g-force. As used herein, PSD profile means information comprising one or more frequencies together with amplitudes and/or power densities thereof.

In some specific embodiments, the vibrations in the two axes are induced simultaneously, i.e. the liquid is vibrated in both directions, either in a part or the entire process. In some embodiments, the liquid comprises a pharmaceutical product. In particular, said pharmaceutical product comprises a biological product, e.g. a protein, antibody, nucleic acid, sugar, or conjugates and combinations thereof.

In some embodiments, the set of instructions for different axes (e.g. X and Z) are different; the difference can be e.g. in the PSD profile, and/or the time duration specified for each of the frequencies which will be applied for inducing vibrations in the liquid.

In some embodiments, the amplitude of vibrations in one axis is proportional (e.g. linearly proportional) to those of the other axis; for example, densities of vibrations in one axis can be about 1.5-10 folds of that in the other axis or axes.

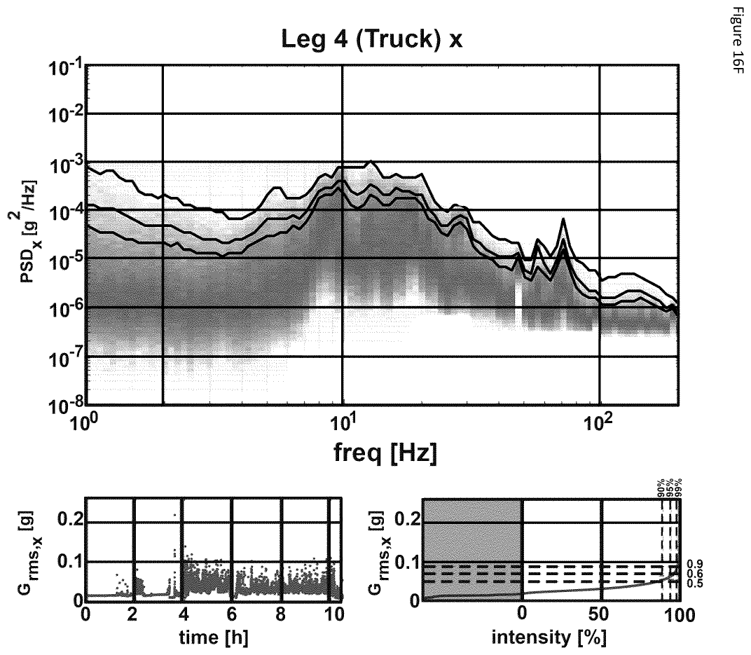

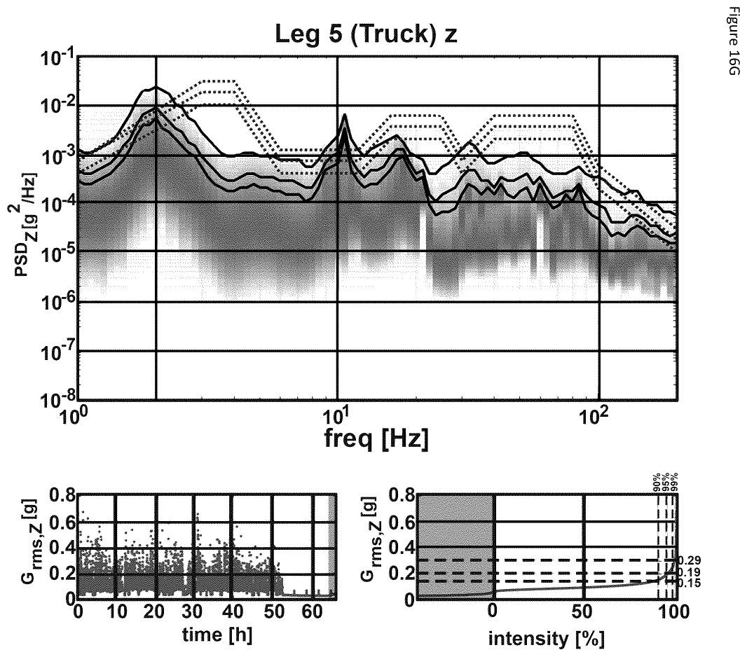

In some embodiments, the PSD profile is designed based on measurement of vibrations in one or more real life shipments (RLS). Said vibrations can be measured e.g. by including sensors in shipment packages in a RLS and recording the frequencies and densities thereof over the shipment period. The densities in the PSD profile can be deduced e.g. by calculating the average of densities recorded by several sensors during RLS. Example 2 provides an exemplary embodiment in this respect. In an embodiment, the PSD profile is as in FIG. 16 (A-G). In an embodiment, the PSD profile is within a 9 dB (decibel) range of solid line or a dotted line in a PSD plots of FIG. 16 (A-G), meaning that, for a given frequency, the intensity will be as indicated by one of said lines, or up to 9 dB (e.g. 1 dB, 2 dB, 3 dB, 4 dB, 5 dB, 6 dB, 7 dB, 8 dB, or 9 dB) less or more than that intensity.

In some embodiments, the PSD profile is according to ASTM D4169 standards, e.g. Air level I, II, or III, Rail level I, II, or III, or Truck level I, II, or III. ASTM standards (Designation: D4169) are published by American Society for Testing and Materials (ASTM) International, and are updated every few years usually (the current ASTM standard is published in 2022, hence denoted as ASTM D4169-22).

In some embodiments, the quality assessment as of the liquid is performed using analytical methods, for example those known to the person skilled in the art and applicable to the particular liquid, e.g. Size-Exclusion Chromatography (SEC), Ion-Exchange Chromatography (IEC), Analytical ultra-centrifugation, visible or sub-visible particle analysis.

In an aspect, provided herein is a two-dimensional shaker adapted to execute the steps of the method of the first aspect described above. By “two-dimensional” it is meant that the shaker has means enabling shaking an object (e.g. a liquid container) in two perpendicular directions simultaneously. Such shaker can be e.g. devised by implementing independent linear motors for moving the liquid sample along each axis. In particular, noise-cancelling methods are applied to the motor controllers in order to omit cross-dependency between movements in different directions. This is achieved e.g. by the use of sensors on each axis in order to detect and counter-act the unwanted vibrations.

In some embodiments, the shaker is able to perform a frequency sweep mode; a sweep mode comprises inducing vibrations starting from one extreme of the frequency range (e.g. the lowest frequency) and gradually increasing the frequency towards the other extreme (e.g. the highest frequency). The sweep mode can be performed in the two axes in parallel mode (i.e. both starting from low frequency or both starting from high frequency) or anti-parallel mode (i.e. starting in one axis from the low frequency and in the other axis from the high frequency). In some embodiment, the vibrations are in the form of constant sine wave (e.g. without sweep mode). In some specific embodiments, the frequency range is between 1-300 Hz.

In some embodiments, the shaker comprises a temperature controlled chamber. This enables maintaining the temperature of the liquid during the procedure in a proper range, e.g. similar to temperatures applied during RLS. For example, liquid formulations comprising biopharmaceuticals are usually kept in a temperature of 2-8 °C. In some embodiments, the temperature in the chamber is kept at 2-60 °C, e.g. 2-8 °C.

In some embodiments, the shaker comprises a vial holder and/or holder(s) for syringes. This enables placing the liquid sample in a variety of containers as appropriate. In some embodiments, the holder is adapted to function with different tilt angles.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, prevail. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### EXAMPLES

### Example 1

In this study, a novel two-dimensional shaker was used to assess the impact of a second dimension in the vibration test on product quality, as two-dimensional vibration is closer to real life shipping. Furthermore, the current measures that pharmaceutical companies adhere to were analysed regarding their protection of drug products from possible extra stress.

Vials were vibrated in either one or two dimensions and some external factors (e.g. temperature, surfactant concentration) were changed to assess their impact. It was found that two-dimensional vibration, contrary to one-dimensional vibration, induces splashing in vials. Two-dimensional vibration also showed more (sub-) visible particle formation than one-dimensional vibration. These results raised concerns regarding drug stability during shipping and an experiment with different concentrations of surfactant was conducted. The results showed that even the smallest concentration of surfactant tested, which was 120 times smaller than the concentration generally used in biopharmaceutical formulations, was sufficient to inhibit (sub-)visible particle formation in one- and two-dimensional vibration. This study formed a first basis for the testing of two-dimensional vibration and served to assist formulation scientists in knowing how their biopharmaceutical products are agitated during transportation and how these conditions are best replicated in-house to test the drug products during development.

### Experimental Procedures

### Real Life Shipping Study

A real life shipping study was conducted in Europe and North America. Accelerometers were placed inside boxes between packaged water filled vials. These boxes were shipped in five stages (Table 2). After shipping, the accelerometers were analysed and Power Spectral Density (PSD) profile and plots were derived from the acceleration measurements.

TABLE 2
Real life shipping schedule.

| | Truck 1 | Truck 2 | Air | Truck 3 | Truck 4 |
|---|---|---|---|---|---|
| Duration (hours; | 0.5 | 4 | 8 | 10 | 48 |
| approximately) | | | | | |

### Monoclonal Antibodies and Formulations

Four different monoclonal antibodies, formulated in buffer were used in this study (mAb1, mAb2, mAb3, mAb4) and their different qualities are shown in Table 3. All formulations were filtered prior to shipping with a sterile filter of 0.22 micrometre pore size (Millex Sterivex® GV) under laminar air flow conditions, closed with a bromobutyl teflonized and siliconized stopper and finally sealed with an aluminium crimped cap. Two vial sizes were tested, 3.2 ml was filled in 6 ml vials and 6.4 ml was filled in 20 ml vials (Fiolax type I glass, Schott). These filling volumes result in equal fill height in vertical orientation, which is beneficial for high speed video analysis as found in previous studies. For the surfactant studies, different percentages of all-oleate PS80 (0.0005, 0.001, 0.0015, 0.002, 0.003, 0.01 and 0.06) were added to mAb1. Vials were stored at 2-8 °C.

TABLE 3
Antibody Overview.

| | mAb1 | mAb2 | mAb3 | mAb4 |
|---|---|---|---|---|
| IgG class | 1 | 1 | 1 | 4 |
| Protein content | 72 | 71.2 | 67.9 | 62.8 |
| pH | 5.5 | 5.8 | 6.1 | 5.8 |
| Extinction | 1.63 | 1.50 | 1.48 | 1.44 |
| Coefficient | | | | |
| (280 nm) | | | | |

### Protein Concentration

For protein concentration measurements, either a SoloVPE Spectrophotometer (C. Technologies) or the UV/Vis-Photometers Lambda35 (Perkin Elmer) were used.

### Vibration Studies

Liquid antibody formulations were agitated using the two-dimensional shaker of the present invention. For the described studies, the ASTM D4169 guideline 2016 Air level I PSD profile was used, either only vibrating in the X- or Z-axis, or in both axes simultaneously. Parameters such as temperature, vial position and vibration time could be adjusted with the shaker. Controls were vials with the same antibody formulation, but were not agitated.

### High Speed Video Analysis

A MotionBLITZ® EoSens mini 1-1 (MIKROTRON) in combination with a Macro 100 F2.8 D AT-X PRO (Tokine) camera lens were used to record high speed video footage of the vibrating vials at 200 frames per second.

### Visual Inspection

A visual inspection was performed using an EP black/white light box. Magnified visual inspection was carried out using a Seidenader V 90-T (Seidenader Maschinenbau GmbH). This method allows visible inspection through rotation by rollers and the inclusion of a 2-fold magnifying lens.

### Light Obscuration

A HIAC 9703+ equipped with a HRLD-150 detector by Beckman Coulter was used. Samples were analysed using four injections of 200 microliter. The average of the last 3 injections was recorded. COUNT CAL 5 μm (3000 Particles per ml) Count Precision Standard from Firma Thermo Scientific (cat. no. CC05) was used as a calibration reference. Between every sample analysis the apparatus was rinsed with water until the ≥2 μm particle counts were ≤20, the ≥5 μm particle counts were $5, the ≥10 μm particle counts were ≤1, the ≥25 μm particle counts were ≤1 and the ≥50 μm particle counts were 0.

### Background Membrane Imaging

Sub-visible particle quantification was also performed with the high throughput method by analysing triplicates with the Horizon instrument (Halo Labs, Burlingame, CA). A 0.4 μm pore size polycarbonate background membrane plate was used and a 40 μl sample volume per well in triplicate. Liquid was removed with vacuum at 200 mbar. Samples above 3% membrane coverage were indicated as oversaturated. HORIZON VUE software version 3.0.0.121 was used.

### Surface Tension

A drop profile analysis tensiometer (PAT1M, Sinterface) was used for surface tension analysis. Analyses were run for 960 seconds (16 minutes) per sample at 22 °C.

### Results

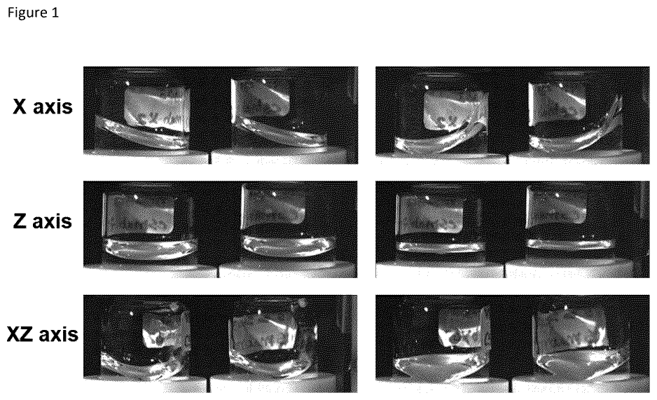

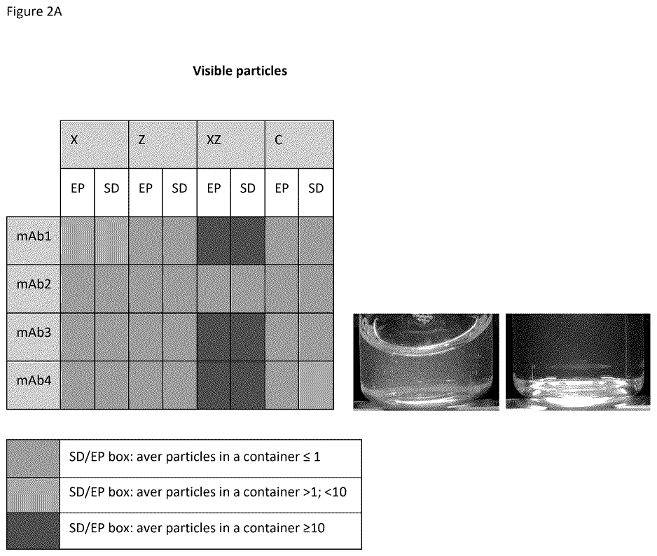

Firstly, it was determined whether there was a difference in wave formation between one-dimensional and two-dimensional vibration. Using the most intense ASTM D4169016 profile (Air level I), different wave formations and splashing patterns could be seen (FIG. 1), whereby the X- and Z-axis showed no splashing and XZ vibration showed splashing and droplet formation.

XZ Simultaneous Vibration Induces More Particle Formation than One-Dimensional Vibration

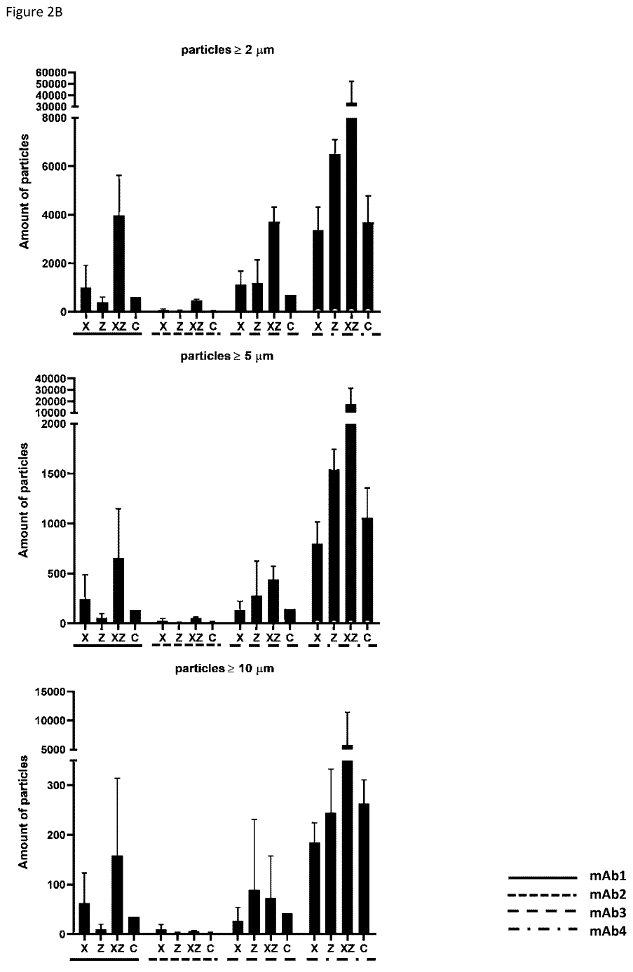

To assess if there was a different effect on product quality between vibrating in one-dimensional or two-dimensional axes (X, Z and XZ combined), vials were placed on the two-dimensional shaker and shaken for thirty minutes with the ASTM D4169-16 Air level I profile at room temperature. This profile was chosen as it is the most severe vibration profile of the ASTM D4169-16 profiles and was the most likely to produce particles. Four different antibodies were tested and compared. When observing the results, only the vials subjected to simultaneous XZ vibration had more than 10 visible particles, which resembled a particle cloud (FIG. 2A). In the subvisible particle range, antibodies mAb1 and mAb4 have the highest particle amount for the vials subjected to the simultaneous XZ vibration as well. In the particle range of 2-4 μm we can see a difference of at least 1000 particles per ml. Antibodies mAb2 and mAb3 also have the highest particle count for the particle range of 2-4 μm, but do not uphold this trend in the bigger particle counts (FIG. 2B).

### Possible Decrease in Particle Formation for Formulations Vibrated at 5 °C.

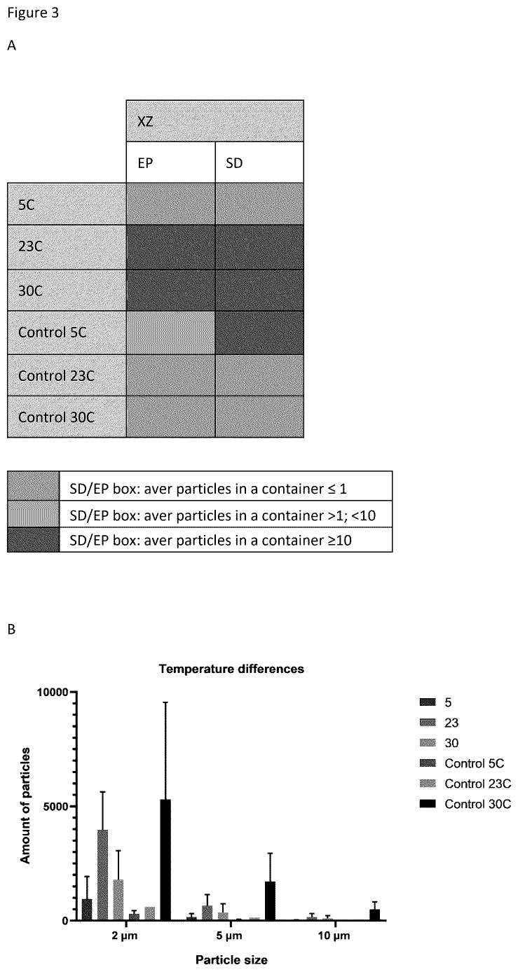

One of the advantages that the new prototype two-dimensional shaker offers, is the ability to adjust the temperature of the vibration chamber. During transportation, biopharmaceuticals are usually kept in a cooling cell between 2-8 °C as higher temperatures are known to accelerate aggregation pathways [Nowak, C.; K. Cheung, J.; M. Dellatore, S.; Katiyar, A.; Bhat, R.; Sun, J.; Ponniah, G.; Neill, A.; Mason, B.; Beck, A.; Liu, H. Forced Degradation of Recombinant Monoclonal Antibodies: A Practical Guide. MAbs 2017, 9 (8), 1217-1230]. With this experiment, it was aimed to see if the harsh agitation of XZ-axis vibration is influenced by lower and higher temperatures for mAb1 formulations. Sub-visible particle formation seems to be lowest in the 5 °C agitated vial, its control and the 23 °C control. The highest sub-visible particles can be seen in the 30 °C control and the 23 °C agitated vial (FIG. 3B). Interestingly, the 5 °C control showed more particle formation in the visual inspection than its agitated version, which is not reflected in the light obscuration data (FIGS. 3A and 3B). Similarly, the 30 °C control did not show visible particle formation, but high sub-visible particle formation.

### Difference in Particle Formation Between Vial Sizes and Orientations

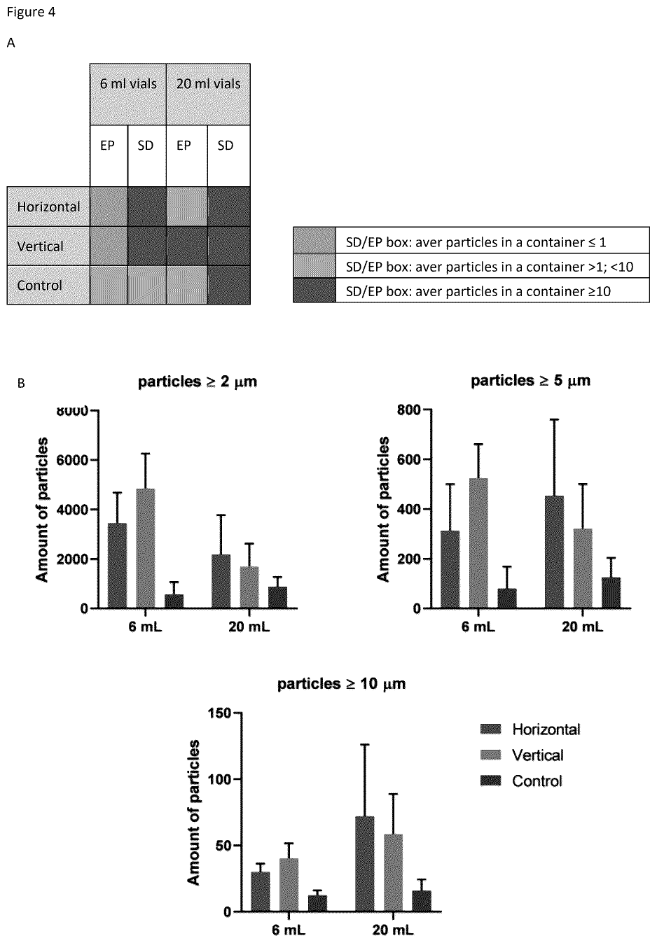

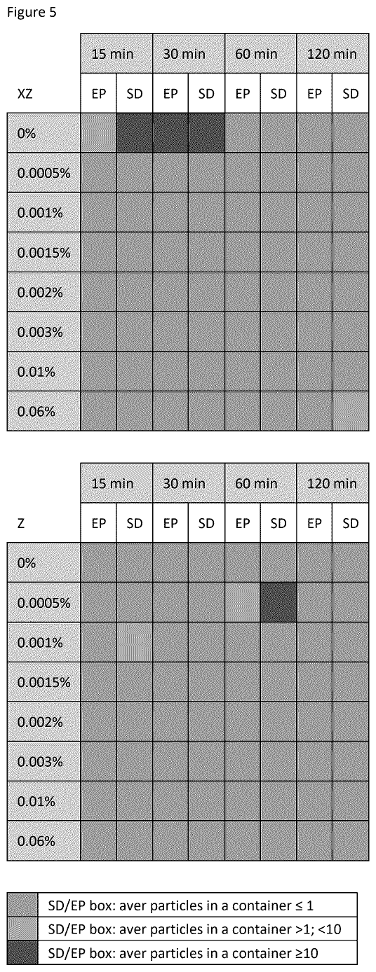

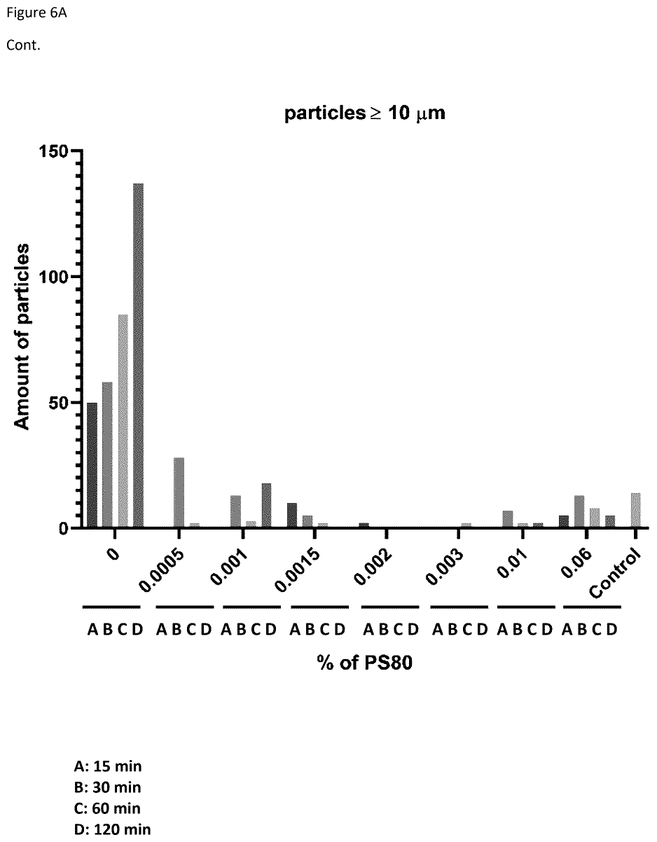

Previous research has indicated that the orientation of vials when being shaken, influences the shear stress distribution in vials [Bai, G. et al, 2012, DOI: 10.1016/j.ijpharm.2011.11.044]. With this experiment, it was aimed to investigate which effect horizontal versus vertical orientation had on (sub-)visible particle formation and if different vial sizes would show additional effects for mAb1 formulation. The vials were shaken once more with the two-dimensional ASTM D4169-16 Air level I profile at room temperature. Almost all vials show some particles in the visual particle inspection (FIG. 4A). Only the horizontally and vertically agitated 6 ml vials showed at most one particle in the black and white box, but more than ten particles under the Seidnenader analysis. From the gathered results it was mostly notable that the different vial sizes showed opposite effects when it comes to orientation. The 20 ml vials show more sub-visible particles in the horizontal position and the 6 ml vials show more sub-visible particles in the vertical orientation (FIG. 4B).

### Polysorbate 80 Reduces Particle Formation in One- and Two-Dimensional Vibrated Liquid Formulations

To assess whether already in use pharmaceutical approaches for particle prevention are relevant for one- and two-dimensional vibration stress, mAb1 formulated in buffer was mixed with different concentrations of Polysorbate 80 (PS80) (Table 4). The formulations were supposed to have approximately the same protein concentrations, but due to a calculation error the 0.06% PS80 has a much lower concentration. These different formulations were then agitated in the XZ- and Z-axis for either 15, 30, 60 or 120 minutes at room temperature. Subsequently, the formulations were analysed for (sub-)visible particles using visible particle analysis, light obscuration and background membrane imaging. The visible particle analysis showed more than 10 particles in the 0% PS80 formulations that were shaken for 15 and 30 minutes in the XZ-axis (FIG. 5) and in the 0.0005% PS80 formulation shaken for 60 minutes.

TABLE 4
The different concentrations of PS80 and the final
in formulation CEA protein concentrations.

| PS80% (w/v) | Protein concentration (mg/ml) |
|---|---|
| 0 | 72 |
| 0.0005 | 72 |
| 0.001 | 72 |
| 0.0015 | 72 |
| 0.002 | 71 |
| 0.003 | 71 |
| 0.01 | 65 |
| 0.06 | 30 |

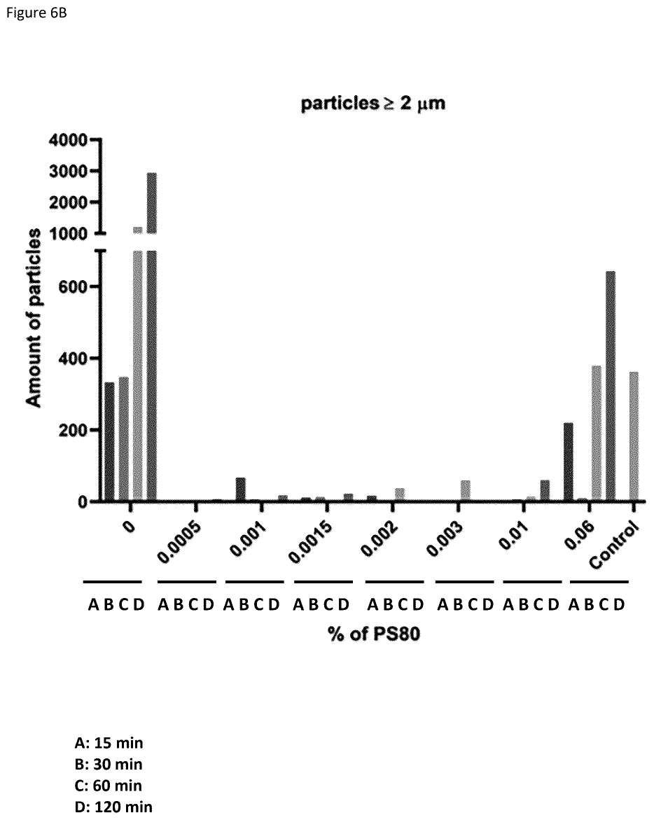

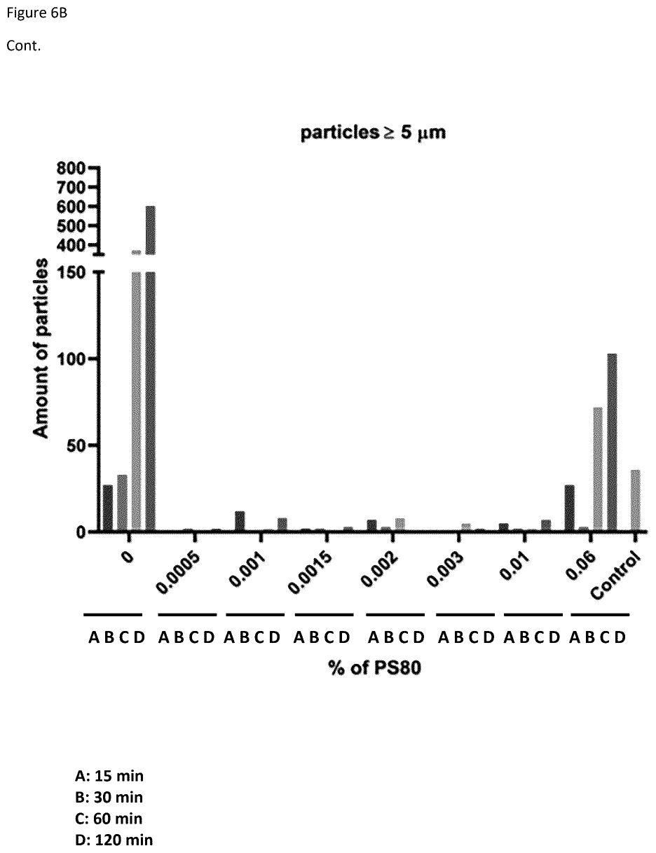

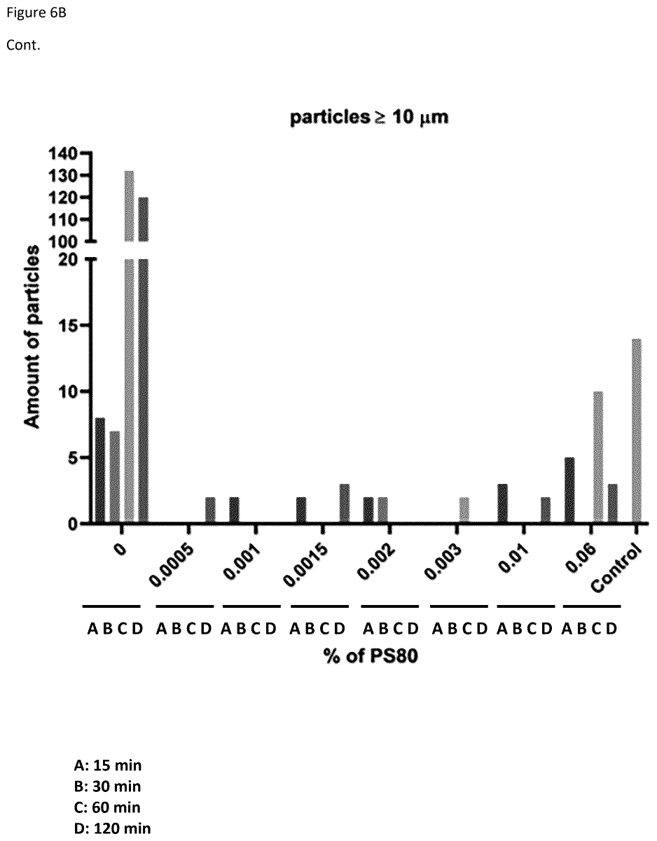

When looking at the light obscuration sub-visible particle analysis, the amount of particles in the formulations without surfactant is higher than in the formulations with PS80 (FIGS. 6A and 6B). There also seems to be a time-dependent increase in particles where the formulations without surfactant are concerned, where longer vibration times lead to higher particle amounts. Interestingly, in the Z-axis agitation, the 60 and 120 minute shakes seem to have had a much larger impact on particle amounts than the 15 and 30 minutes.

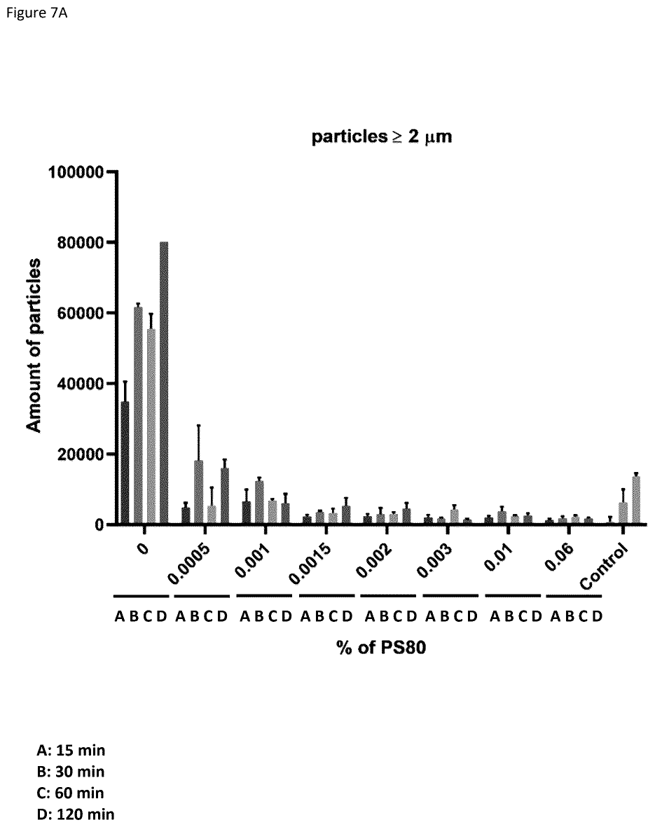

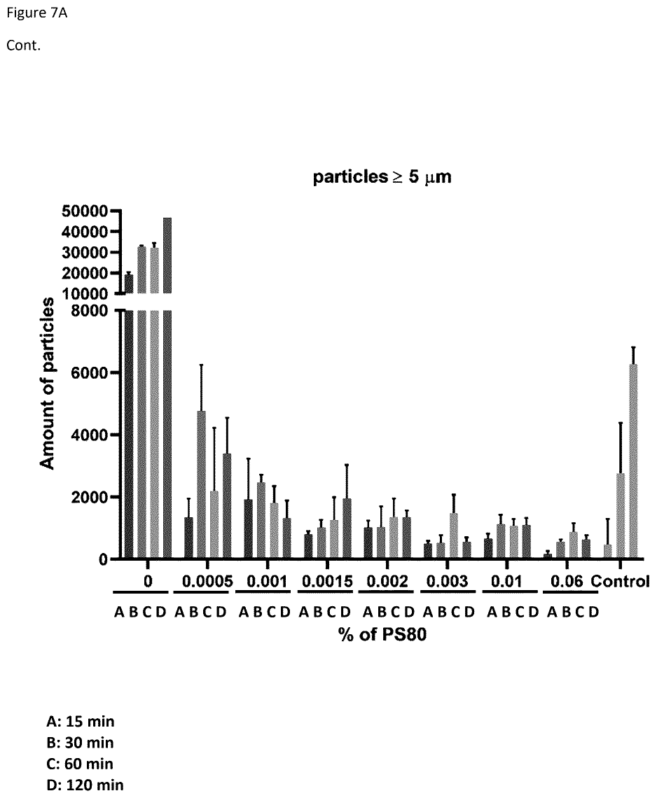

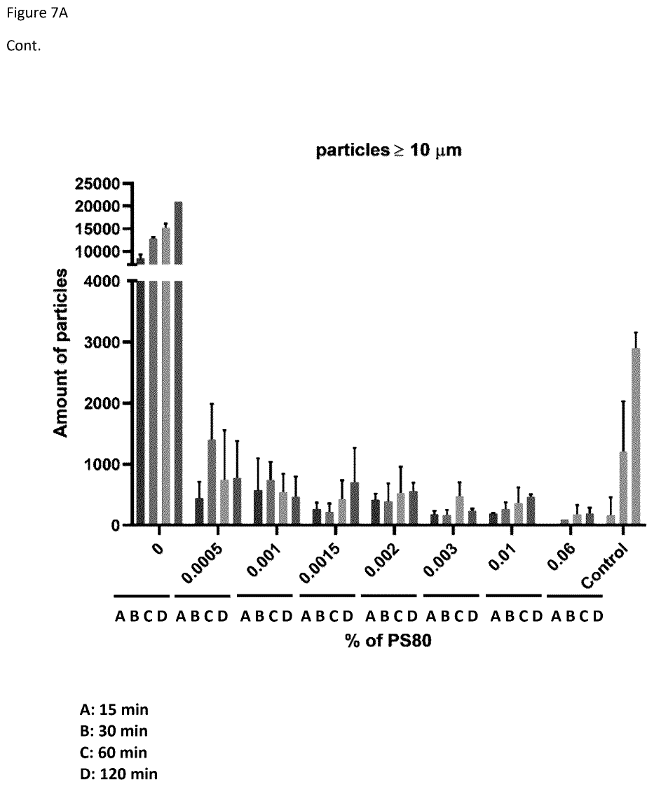

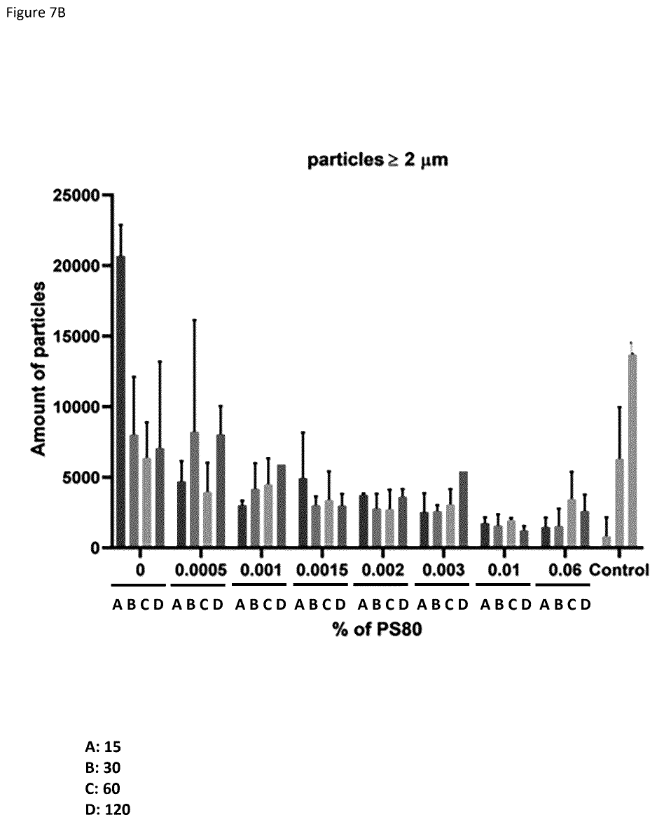

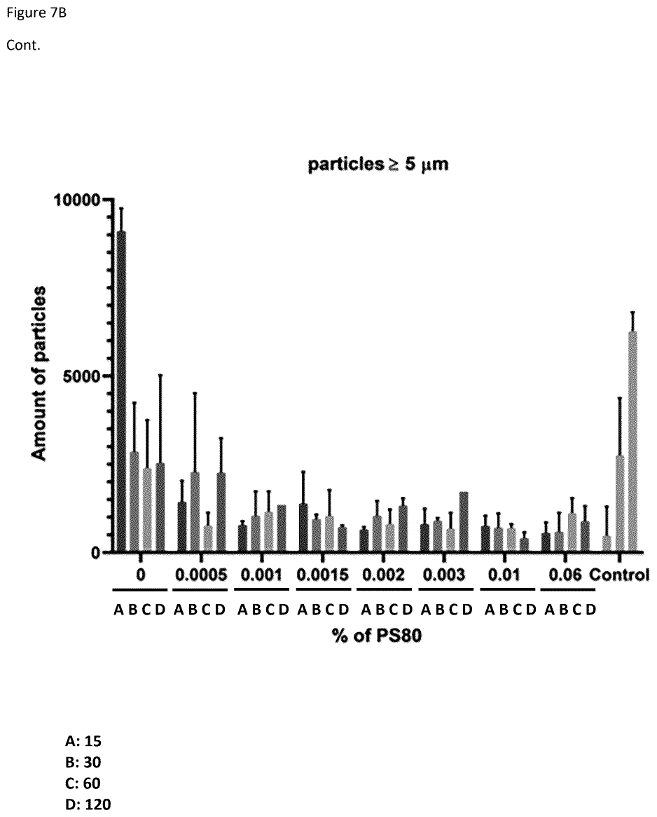

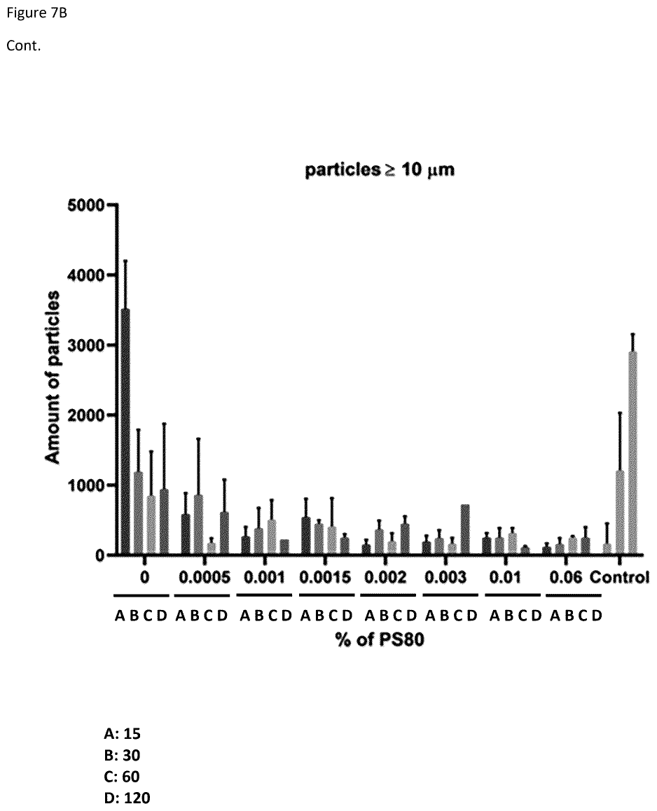

The results of the background membrane imaging results also depict a decrease in sub-visible particles when surfactant is added to the formulation for the XZ-axis agitation (FIG. 7A). In the Z-axis agitation, however there seems to be a higher number of particles in the 0% PS80 formulation agitated for 15 minutes, but there seems to be less significant difference between the rest of the formulations (FIG. 7B).

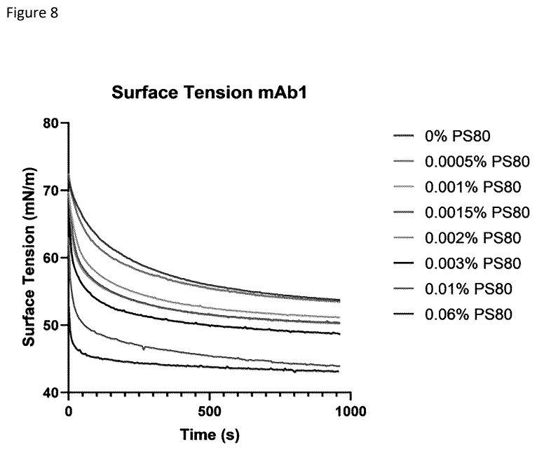

Previous research has shown that there is a correlation between surface tension and aggregation/particle formation. Using a drop profile analysis tensiometer, the surface tensions of mAb1 with different concentrations of PS80 as used in the previous experiment was identified. As can be seen in FIG. 8, higher concentrations of PS80 give a lower surface tension.

### DISCUSSION

The objective of this experiment was to gain more knowledge about transportation stresses and to determine whether the current preventive measures during formulation development are sufficient for preventing product quality impact. The 2016 ASTM D4169 Air level I profile was used for this study as it is the most severe profile and was sure to give some particles amounts which could be compared.

In this study, it was first looked into the difference between one-dimensional and two-dimensional liquid agitation through vibration. The results seem to indicate that simultaneous XZ vibration induces more particle formation than one-dimensional vibration. This is most likely because of the splashing that was seen during the high speed video analysis. Previous studies investigated that splashing in liquid formulation caused high shear stress and therefore protein aggregation [Koepf, E. et al, DOI: 10.1016/j.ijpharm.2017.12.043; Maa, Y. F.; Hsu, C. C., 1997, DOI: 10.1002/(SICI) 1097-0290 (19970620) 54: 6<503::AID-BIT1>3.0.CO;2-N; Das, T. K. et al, 2021, DOI: 10.1016/j.xphs.2021.09.030, Zubiaga, A. et al, 2019, DOI: 10.21152/1750-9548.13.1.61]. Internal simulation research showed that high local peak shear is expected upon droplet impact to liquid, whereas wave formation leads to lower overall shear in the liquid, which is in-line with the observations of particle formation in this study [Hostettler, M.; Brunner, D.; Rosenthal, F.; Clemens, M.; Koepf, E.; Boiger, G. K. Analysis of Falling Droplets into Resting Liquid and Resulting Shear Stresses. In International Conference of Multiphysics, Online; 2020]. In FIG. 2 it is shown that especially the particles that were determined by light obscuration to be in the 2-4 μm range, increase when exposed to XZ vibration more than one-dimensional vibration. Even mAb2, the stable reference molecule, seems to have more particle formation under XZ vibration.

The novel shaker is more representative compared to established transportation simulation, which is operating under ambient conditions. Our results indicate that temperature has an effect on the formation of (sub-)visible particles. Previous research shows that accelerated aggregation and denaturation happens over 50 °C [Menzen, T., Friess, W., 2014, DOI: 10.1002/jps.23827; Vermeer, A. W. P., Norde, W., DOI: 10.1016/S0006-3495 (00) 76602-1], however our results seem to indicate that there is also a difference between lower temperatures such as 5 °C and 23 °C. This is not fully surprising, as temperature can change the viscosity of a liquid and this subsequently influences the shear stress that liquid experiences [Woldeyes, M. A. et al, 2020, DOI: 10.1021/acs.molpharmaceut.0c00552; Iqbal, M. J.; Chaudhry, M. A., 2009, DOI: 10.1016/j.jct.2008.09.016]. Finally, there have also been reports where room temperature was considered the worst case scenario, as companies use to ship their drug products in temperature controlled chambers [Ammann, C., 2011, DOI: 10.1208/s12249-011-9684-0; Fleischman, M. L. et al, 2017, DOI: 10.1016/j.xphs.2016.11.021].

For the horizontal versus vertical vial orientation experiment, the visible particle inspection does not completely line up with the sub-visible particle analysis. This could be due to there being more sub-visible/smaller sized particles in the agitated vials and more visible particles in the controls. The fact that more sub-visible particles could be seen in the 6 ml vials in the vertical orientation and in the 20 ml vials in the horizontal orientation could be explained by the difference between air-liquid interfacial stress and shear stress [Koepf, E. et al, DOI: 10.1016/j.ijpharm.2017.12.043; Maa, Y. F.; Hsu, C. C., 1997, DOI: 10.1002/(SICI) 1097-0290 (19970620) 54: 6<503::AID-BIT1>3.0.CO;2-N; Narhi, L. O. et al, 2022, DOI: 10.1016/j.xphs.2022.01.011]. In the horizontal position, the formulation in the 20 ml vials had a bigger air surface and splashed against the bottleneck, which is where Bai et al. found the most shear stress occurs in a vial [Bai, G. et al, 2012, DOI: 10.1016/j.ijpharm.2011.11.044]. The 6 ml vial in horizontal orientation on the other hand did not splash against the bottleneck, but rather the middle of the stopper. In the vertical position, the 20 ml vial did not really show any splashing and the 6 ml vial, as seen in FIG. 1, does. Finding a simple head-to-head comparison for this research questions is not straight forward, due to the complexity of interactions between fill volume, fill height and the impact of the vial geometry dependent on the orientation of the vial. The chosen fill volumes are ideal for comparison of vertical vibrations as with preliminary image analysis studies. A set-up with ranges of filling parameters in a follow up study would allow for more detailed investigations. Furthermore, leached silicon oil from the stopper can lead to higher sub-visible particles when in contact with product solution as it was the case with the 6 ml vial compared to the 20 ml vial [Grapentin, C. et al, 2020, DOI: 10.1016/j.xphs.2020.03.010; Pavanetto, F. et al, 1991, 10.1016/0378-5173 (91) 90234-F; Sendo, T. et al, 1995, DOI: 10.1002/jps.2600841218]. Fourier-transform infrared spectroscopy (FT-IR) analysis would give information about the composition of the particles and could help us understand their origin.

To prevent protein aggregation, formulation developers add excipients, such as surfactant and sugars to the drug product formulation. In this study PS80 was used, because PS80 is said to offer higher protection against agitation-probed aggregation [Singh, S. M. et al, 2017, DOI: 10.1016/j.xphs.2017.08.011]. In the results show that the amount of (sub-)visible particles in formulations with surfactant are much lower than in the formulation without PS80. In the BMI results, there also seems to be a correlation between the amount of surfactant and the amount of particles. This is expected, as the surface tension measurements show a lower surface tension with higher concentrations of PS80 (FIG. 8) and this is in line with previous research [Das, T. K. et al, 2021, DOI: 10.1016/j.xphs.2021.09.030; Kannan, A. et al, 2021, DOI: 10.1016/j.xphs.2020.10.036]. However, the protein concentration mAb1 was almost half the target concentration (72 mg/ml) in the 0.06% PS80 formulation due to a calculation error and the light obscuration measurements do not follow the same trend.

It should be noted that the light obscuration is a noisy method and there can be large discrepancies between samples that have been treated the same way. For this reason, product quality analysis is not limited to sub-visible analysis and visual inspection, but also other stability indicating methods such as size-exclusion HPLC. As light obscuration gives diverse results, only the particles up to a size of 10 μm are shown in the light obscuration results. Background membrane imaging is a method also used for the measurement of sub-visible particles and has some advantages over light obscuration, but also disadvantages [Vargas, S. K. et al, 2020, DOI: 10.1016/j.ijpharm.2020.119072]. Therefore it is used in this study alongside light obscuration.

Although this study showed interesting results, such as the increase of (sub-)visible particles in two-dimensionally agitated liquid formulated biopharmaceuticals, it was also good to see that the addition of surfactant significantly decreased the formation of (sub-)visible particles and therefore the preventions that formulation developers take against aggregation and particle formation are effective. Using a two-dimensional shaker as disclosed herein could be extremely useful for formulation scientists and pharmaceutical companies to know how their biopharmaceutical products are agitated during transportation and how these conditions are best replicated in-house to test the drug products during development.

### Example 2

### Scope

Product Quality Impact (PQI) studies are performed for large molecule products, such as antibody drugs, to evaluate the impact of commercial shipping conditions on the quality attributes drug product through representative shipping routes. The main purpose of this study is to record and analyse vibrations and shocks that occur during real life shipment, and how they propagate throughout the stacked product on the pallet. These shipping stresses will be recorded by accelerometers along a representative supply chain for EU/US distribution.

This study provides a better understanding of long-term vibrations within the packaging and their orientation. From simulation and experiments, it is know that horizontal and vertical vibrations do have different impact on the motion of the fluid and therefore different contribution with regard to the stress of the drug (Zubiaga et al., 2019).

Three different study drugs, Active 1, Active 2, and Active 3 are shipped alongside the accelerometers and analysed for potential quality impact of shipment on a drug product (DP). This will allow a direct correlation between an agitation stress profile recorded and the respective product quality impact. The aim is to compare existing model systems regarding the product quality impact and develop novel lab-based approaches to replace real life shipping studies.

This collected knowledge allows to test products under more realistic i.e. representative transport conditions in the laboratory. Such lab-based model systems will help to save costs, resources and the environmental footprint of real life shipping studies. Further insights in the damping and resonance effects of a realistic shipping configuration (e.g. stacking of boxes) will enable those in the art to consider these effects by adjusting the resulting model-system conditions and parameters. Furthermore, data foundation to study the agitation of the drug product vial in its secondary packaging is collected as well.

Drug Product selection

This study assesses the impact of commercial shipping conditions on the quality attributes of drug product. Next to water-filled vials to fill up the boxes and to place the accelerometers, three different drugs will be used for this shipping study (Table 5).

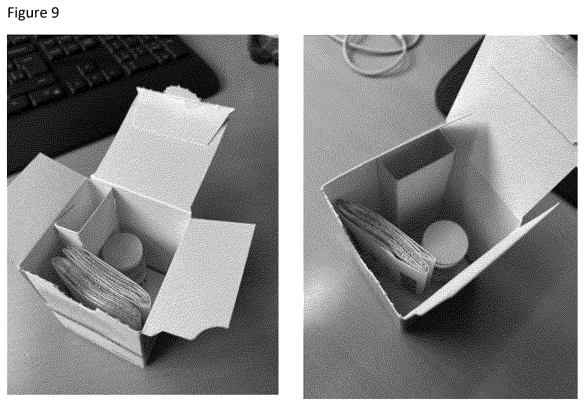

TABLE 5
Drug Product information. The formulations contained
antibodies in concentration range of 25-180 mg/ml, 10-
200 mM of suitable buffers as used for antibody parenteral
formulations in the art, and a pH of 5.5-6.0.

| Description | Active 3 | Active 1 | Active 2 |
|---|---|---|---|
| Formulation | mAb3, 0.01% | mAb1, 0.01% | mAb2, 0.01% |
| | PS20 HP, | PS20 HP, | PS20 HP, |
| | water for | water for | water for |
| | injection | injection | injection |

| | |
|---|---|
| Fill Volume | 2.7 mL |
| Primary | 6 ml vials, colourless, 20 mm (10011673) |
| Packaging | D777-1 serum-stoppers, 20 mm (10067882) |
| Configuration | Flip-Off Seals, 20 mm (10011665) |
| Secondary | Foldingbox, Single Leaflet (FIG. 9) |
| Packaging | |

### Rationale for Drug Product Selection

These formulations have a different susceptibility to product quality changes upon agitation stress. The fill volume and vial configuration are kept identical for all products, to exclude headspace-to-fill volume ratio and surface-to-fill volume ratio as potential influencing factors. ACTIVE 1 and ACTIVE 2 are known to be sensitive to interface-related aggregation. In contrast, Active 3 is assumed more stable upon shaking. The formulations of ACTIVE 1 and ACTIVE 2 as described herein contain only a minimal amount of surfactant. Therefore, these formulations are more likely to show relevant quality changes upon agitation.

The goal with shipping these drug product formulations is to assess a potential impact of agitation stress during representative shipping on DP quality. Interpretation of study results will be performed by comparing stresses of shipped samples and control samples. Therefore, the materials shall be sub-divided into shipped samples and non-shipped control samples. The control samples will be stored under recommended storage conditions in a controlled environment at 2-8 °C. All samples and controls will be visually inspected for the absence of any visual particles prior shipment. The DP is analysed after return. Water-filled vials serve as a filler for the shipping boxes, which enables a realistic positioning of the accelerometers and DP. The water-filled vials will not be analysed.

### Packaging Configuration

### Shipping Boxes (Tertiary Packaging)

All vials and accelerometers is placed in shipping boxes of representative configuration. One shipping box contains 105 secondary packaged vials.

20 vials of each lab-scale produced DP, meaning a total of 60 samples, are included into the shipment. The DP vials will be distributed among the boxes containing accelerometers. The remaining space of each of these shipping boxes containing the DP samples is filled up with water-filled vials. All other shipping boxes are exclusively packed with water-filled vials.

### Pallet Configuration

A total of 24 shipping boxes are placed onto the pallet, with 3 layers of 8 secondary packaging boxes (corresponds to the maximum allowed height of the pallet within the isolated container). Consequently, the pallet will therefore contain a total of 2510 secondary packaged vials (24 boxes à 105 vials→2520 vials; minus 10 loggers→2510). Of which 2450 are water-filled vials and 60 are DP vials. The whole pallet is transferred in a thermal shipping system for airfreight, e.g. RKN e1 or Skycell 1500C. The pallet is placed at the bottom of the plane.

### Agitation Investigation

### BACKGROUND

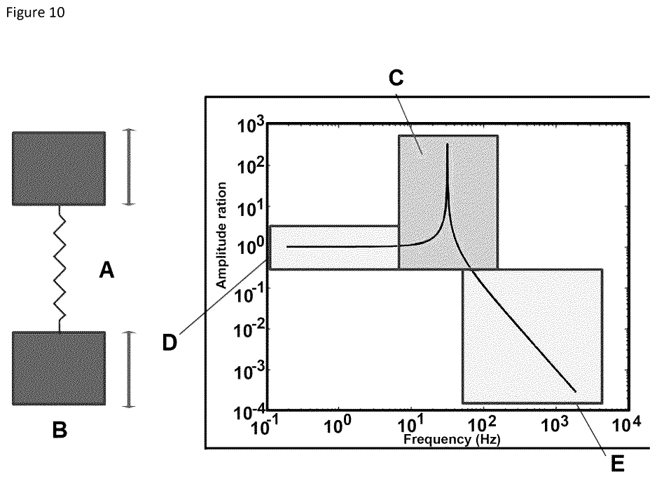

A wave changes as it travels through a medium, the accelerometers serve to measure the vibration intensity at different locations within the pallet. Good practice examples are weighing tables, where a large marble block sits on elastomers. This system results in a very low resonance frequency. For such a system, any wave below this resonance frequency experiences “rigid transmission”, i.e. the wave does not change when travelling through the elastomers. If the excitation is at the resonance frequency, the vibration amplitude increases when travelling through the system (unless the system is above critical damping). Weighing tables systems are designed in a way that all vibration frequencies are above the resonance frequency. In this case, the vibration intensity is significantly reduced, and the measurement is somewhat isolated from external vibration. This system can be considered as a “mass-spring system”, which is shown in FIG. 10 with its three coloured areas (rigid transmission, vibration amplification, wave damping).

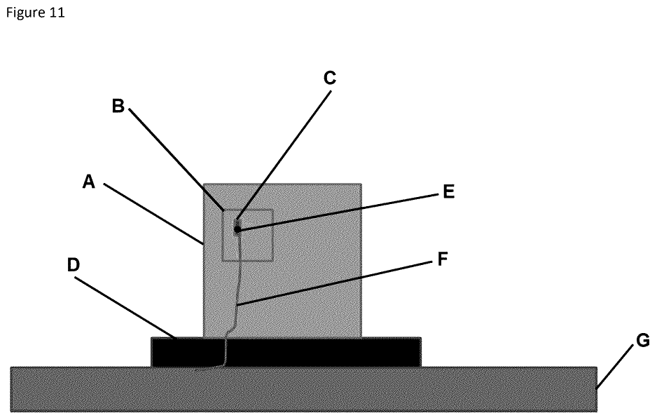

Similarly, the transport box can be seen as mass-spring system; however, it has essentially infinite degrees of freedom. Since there are most probably resonance frequencies below or within our range, it is expected that the packaging influence the vibration intensity at the vial (FIG. 11).

When considering vibrations during transport there are typically two aspects, namely: shocks and long-term vibration intensity. For many applications, shocks are far more important because they exert a high stress to a structure, which can result in its failure. General vibration on the other hand, create a long-term load, resulting in fatigue.

When considering fluids, shocks might be less important because there is not a structure to be destroyed. A shock can result in short intense motions but can have little impact over time on the drug. Long-term vibrations on the other hand can potentially cause constant motion and frequent splashing and thus stress on the drug, which can result in its degradation. It is known that horizontal and vertical vibration do have different impact on the motion of the fluid and therefore different contribution with regard to the stress of the drug (Zubiaga et al., 2019). Therefore, it is important to characterize the stress input and propagation within our shipping system.

### Agitation Measurement

Two distinct objectives shall be completed with the agitation measurement. Firstly, a complete time series of 3-axis acceleration data in a 1600 Hz resolution over the whole course of the shipment is obtained. Furthermore, the second objective is to understand the influence of the packaging on the vibration down to the level of the secondary packaging, in which each vial is packed.

The accelerometers mounted on the lowest level serve as reference sensors, which measure the excitation vibration of the pallet with enough precision for the purposes of this investigation. Lab studies have shown that the measurement on the lowest level within the packaging configuration is comparable to the vibration of the pallet, which is lying underneath. Due to the limited battery life and storage capacity of each logger, five reference accelerometers will start consecutively to cover a maximum span of 15 days.

To reach the second objective, e.g. to quantify changes in vibration intensity, accelerometers are placed at different locations within the pallet. The reference measurement will be compared to the measurements of the remaining probe sensors, which are placed at five different locations within the pallet. The reasoning of taking multiple measurements is that the path of the wave is obviously different for a secondary packaging close to the bottom in comparison to one near the top. Comparing the data from reference to the probe sensors helps understand and determine the vibration transfer from the transport vessel to the secondary packaging through the pallet. This vibration transfer is characterized by a frequency-dependent vibration intensity ratio, which is the amplitude ratio of the probe-measurement to the reference measurement.

The probe sensors record in an interval of 2 minutes recording and 8 minutes pause to cover an identical span. Since frequency spectra are compared this is sufficient to characterize the transmission of the vibration.

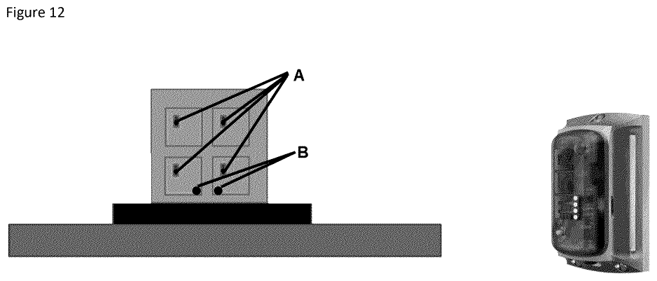

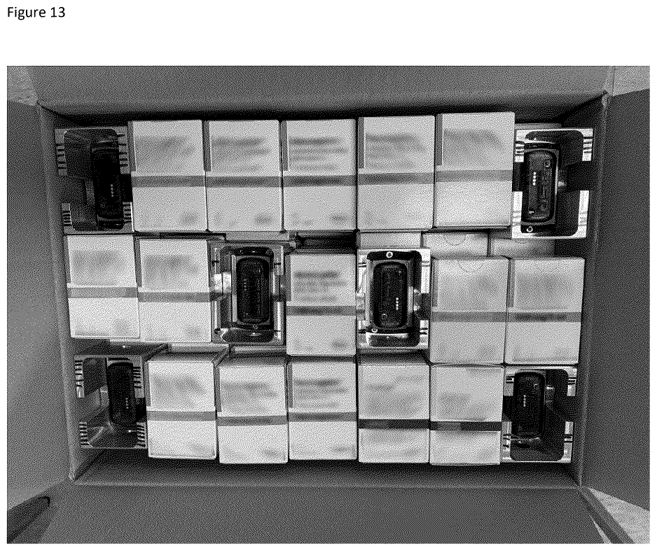

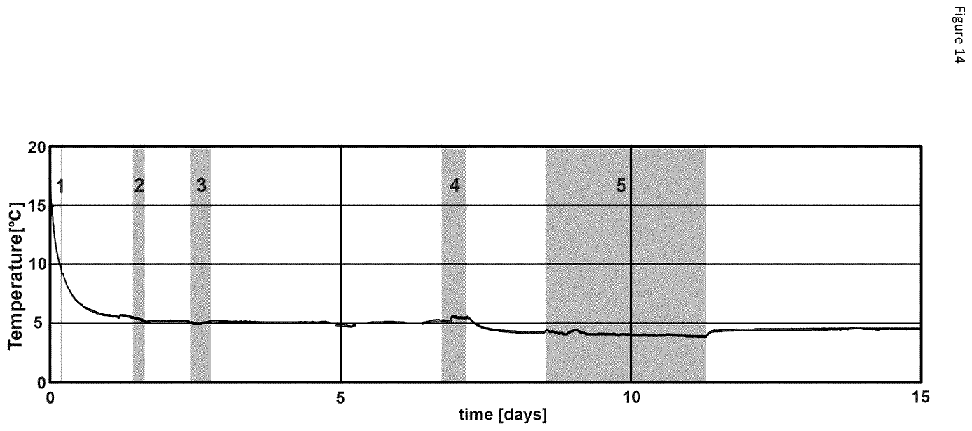

FIG. 12 conceptualizes the experimental set-up where the reference measurement is taken on the lowest level (black) and the remaining probe sensors at different positions within the transport boxes. The accelerometer (sensor, logger and battery within device) is shown in the right side of the figure. It is important that the pallet and the boxes be handled as representative as possible while still staying within the recording period of the accelerometer. Any special treatment can lead to miss-interpretation of the obtained data.

### Accelerometer and Logger Holder

In total 10 MSR data loggers are used to record agitation stress during the entire real-life shipment process. The accelerometers will sit in a custom-made logger holder, which has the same external dimensions as the secondary carton package of a 6 ml vial. The holders are milled from solid aluminum and the accelerometers are secured to it by three M3 machine screws. The accelerometer holder is designed as such, that it allows for “rigid” transition within the frequency spectrum of interest. This means that there are no resonance frequencies below 500 Hz. This ensures that we are only measuring the external vibrations without the holder itself influencing the measurement.

### Drug Product Sample and Sensor Position

Based on the rationale for positioning as explained above, the reference accelerometers are placed as close as possible to the center of the pallet without being in a corner of a box or in direct contact with each other. The probe accelerometers are distributed to cover the whole pallet and an edge. The DP vials are added to the same boxes as the accelerometers, firstly to have a direct measurement of the vibrations within that box, and additionally to consider operational aspects of the packaging procedure and accelerometer activation. This leads to the following allocation of accelerometers and DP vials:

Box 8, 10, 13, 23 and 24 contain three vials of each DP and one probe accelerometer; Box 7 contains five vials of each DP and two reference accelerometers; Box 2, 3 and 6 contain one reference accelerometer.

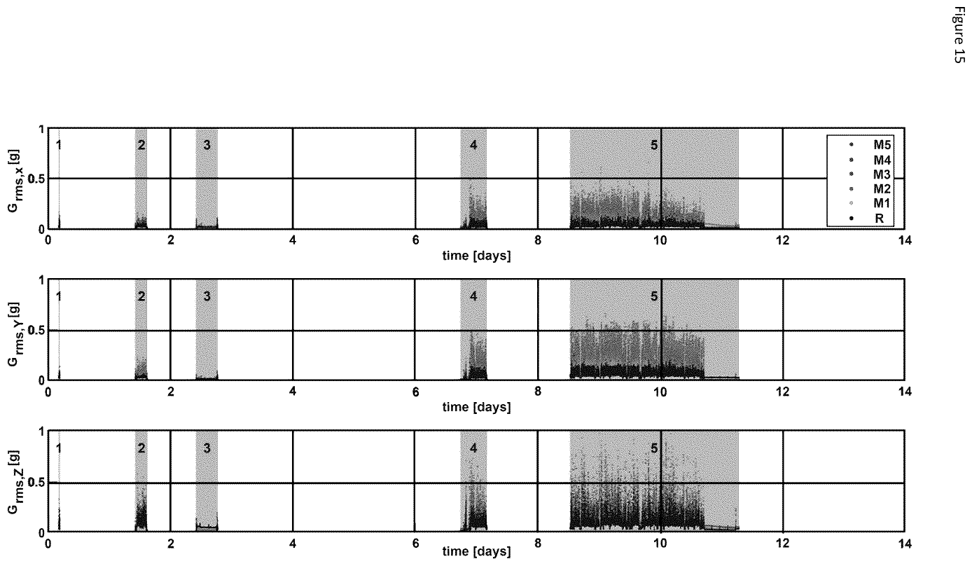

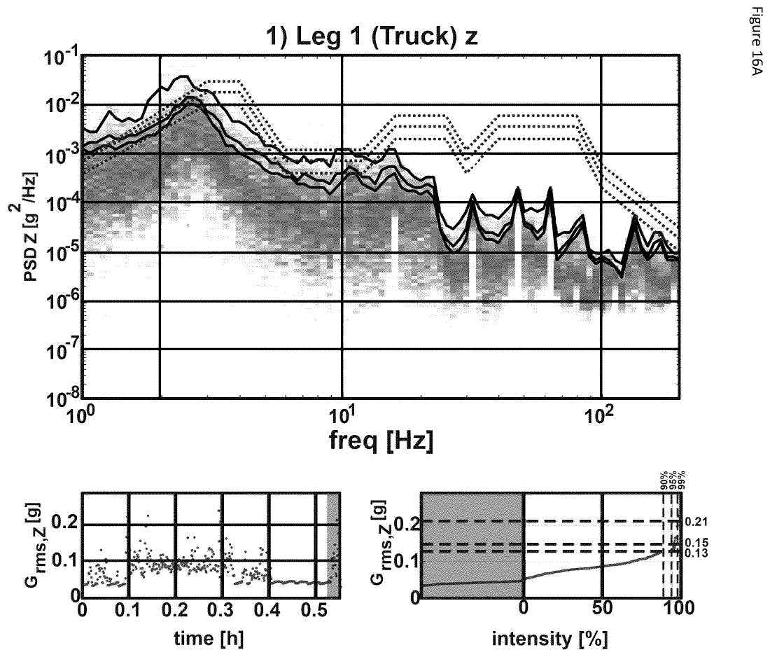

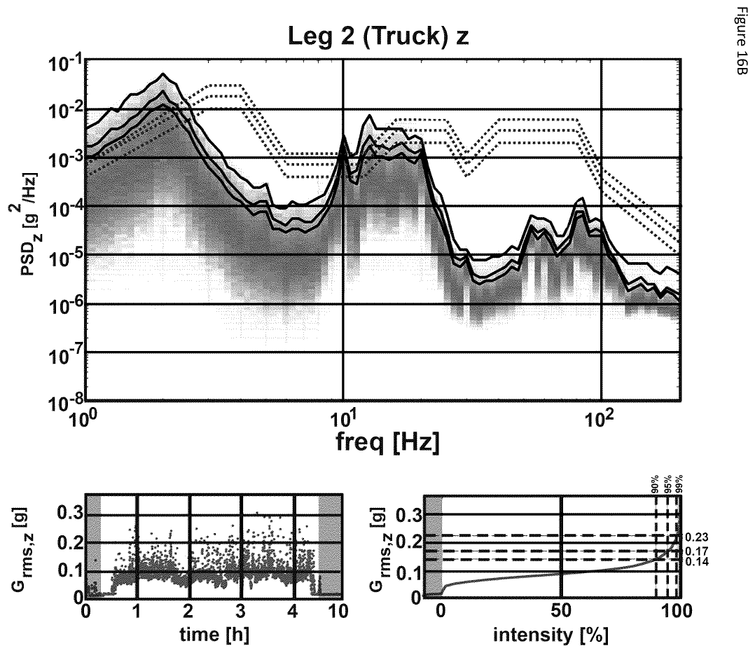

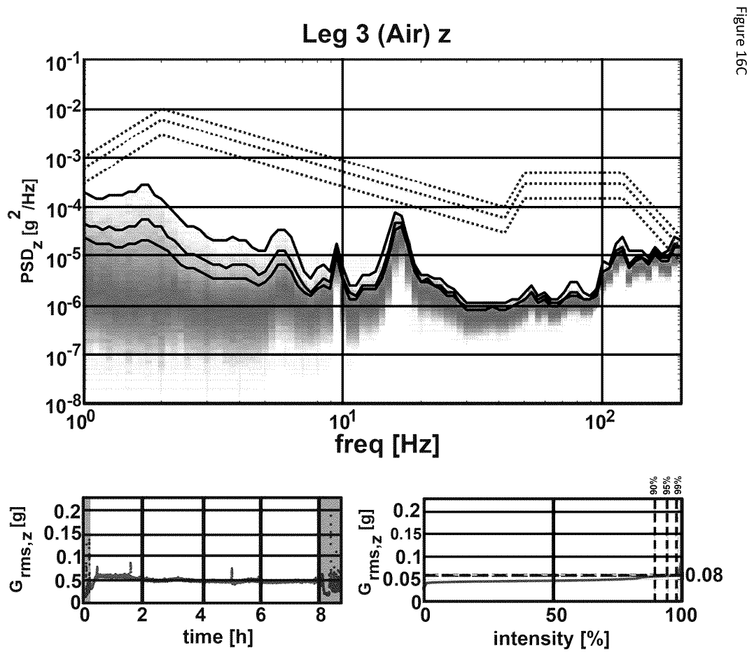

The holder with the accelerometer contained within are placed in the same manner, as a normal secondary packaging with vial would be. The precise location of each sensor within the pallet is defined (FIGS. 13 and 14). FIG. 13 shows an exemplary picture of the accelerometers in the holders within a shipping box packed with secondary packaged vials.

### Real Life Shipment Study Procedure

All vials incl. accelerometers placed at dedicated positions were subjected to real shipping conditions. All DP samples have to be stored at 2-8 °C prior to the start of the shipping study.

The non-shipped control samples were stored at 2-8 °C for the duration of the shipping procedure. Samples were stored at 2-8 °C after return until analysis, and retain aliquots for future analysis can be frozen at −70 °C±20 °C.

The shipping plan was intended to represent a typical shipment of a pharmaceutical product. A qualified thermal shipping system was used to avoid temperature-related impact on the product. The product temperature needs to be kept at 2-8 °C prior shipping, during the entire shipping lane, as well as after shipping until analysis. Each leg of the proposed route covers the following:
Leg 1: transportation by truck; duration: about 45 minutes.
Leg 2: transportation by truck; duration: about 5 hours.
Leg 3: transportation by flight; duration: about 9 hours.
Leg 4: transportation by truck; duration: about 11 hours.
Leg 5: transportation by truck; duration: about 57 hours.

### Product Quality Assessment and Acceptance Criteria

The evaluation of DP quality post-shipment and the respective controls are based on quantitative testing and qualitative comparison.

TABLE 6
Sampling test information.

| | | Testing | | |
|---|---|---|---|---|
| | | Control | Stress | |
| pCQA | Method | sample | sample | Rationale/Comment |
| Size Variants | SE-HPLC | X | X | Physical stresses can result in changes to |
| | | | | size variants |
| Charge | IE-HPLC | X | X | Physical stresses can result in changes to |
| Variants | | | | charge variants |
| Color | Color - HACH | X | X | Shipping stresses can potentially affect the |
| | Lange LICO 500 | | | color of the drug product |
| | or 690 | | | |
| Turbidity | Turbidimeter | X | X | Shipping stresses can potentially affect the |
| | HACH 2100AN | | | turbidity of the drug product |
| Subvisible | HIAC Royco | X | X | The physical stresses encountered during |
| Particles | | | | shipping can result in the generation of |
| | | | | sub-visible particles |
| Visible | Visual Inspection | X | X | The physical stresses encountered during |
| Particles | by EP box | | | shipping can result in the generation of |
| | Extended | X | X | visible particles. Note that the test batches |
| | characterization | | | can already contain particles. Therefore, |
| | (FT-IR) | | | extended particle characterization will be |
| | | | | performed for test and control samples. |
| Protein | UV | X | NT | Although shipping stresses are not |
| Concentration | | | | expected to affect product concentration. |
| pH | pH meter | X | NT | Although shipping stresses are not |
| | | | | expected to affect pH. |
| Surfactant | PS20 content, | (X) | (X) | optional |
| analysis | FFA | | | |

X = analysis, (X) = optional read-out, NT = not tested.

As the DP formulations and configurations used for the study are specifically designed for this study, acceptance criteria are not given. Therefore, results are evaluated according to acceptance criteria for no significant change for each test method (within assay precision). Results of the shipped samples are directly compared to the non-shipped vials as control.

Chromatographic (SE-HPLC and IE-HPLC) profiles of the non-shipped controls and the shipped samples are compared and evaluated regarding a) absence of new peaks and b) overall profile comparability (within assay precision). Moreover, results of the shipped samples are directly compared to the non-shipped vials as control to assess the impact of the shipment on product quality only.

### Results

Temperature was monitored during the transportation. After initial cooling of the sensors and packaging, the temperature remained stable in the expected range (FIG. 14).

### Vibration Monitoring

Vibrations were monitored to understand real-world stress conditions. The overall vibration input to the pallet was measured, and additionally the distribution of the vibrations through the stack of packages was determined.

### Long Term Vibrations

Vibrations were recorded by measuring 3-axis acceleration at 1600 Hz, so 1600 data points per second. To cover the transportation, the five reference loggers at the bottom of the pallet were recording consecutively.

### Total Vibration Energy

The Grms (g-force root-mean-square) defined as the area under the PSD curve measures the overall energy input through random vibration.

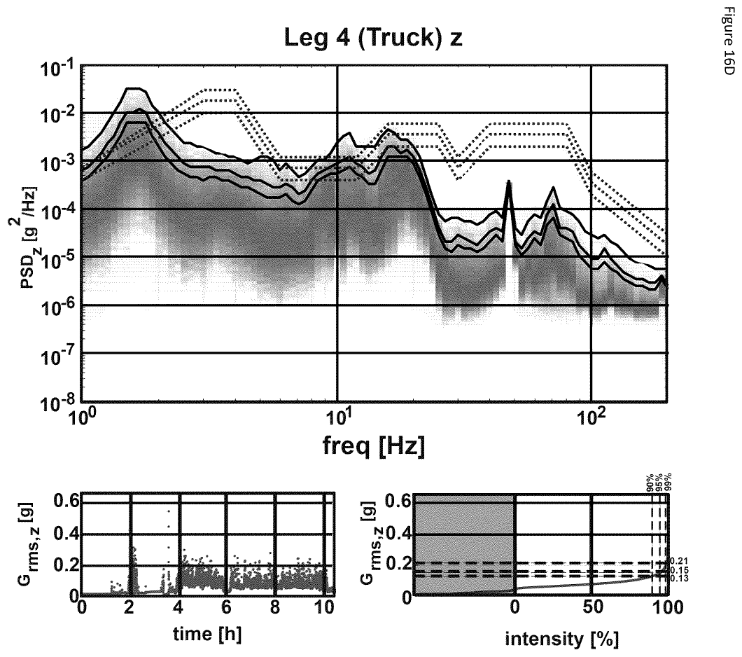

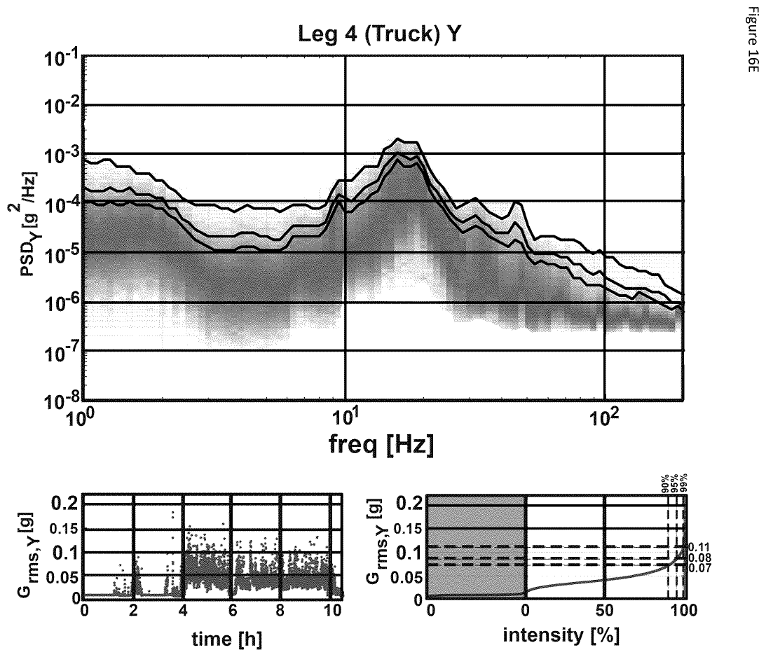

The time series shows that the vibrations occurred during the legs of the transport (1-5). Vibrations were more intense during truck transport (leg 4 and 5) compared to the flight (leg 3). Furthermore, loggers at different height within the pallet show that overall energy was highest at the top (M5) and had a decreasing order down to the reference logger (R) at the bottom of the pallet (FIG. 15). This indicates that vibrations are amplified through the stack of packaging.

The overall vibration intensity was lower than typical vibration testing guidelines recommend such as ASTM D4169-16.

### Spectral Density of Vibrations

To analyse the power spectral density (PSD) of the vibrations, intervals of 2 minutes were combined, fast-Fourier transformed (FFT). The PSD curves describes the random vibration spectral density, e.g. at which frequency what level of energy occurs during that 2-minute interval. All the combined 2-minute interval PSD curves were plotted as density. The density shows the distribution of the intervals. From the density the quantiles and corresponding PSD curve can be derived, e.g. the 10% or 5% worst vibrations in the same fashion as it is for ASTM D4169 guidelines.

The PSD distribution was plotted and the colour gradient shows the quantile or frequency of such a PSD curve to occur. The darkest area is the most frequent e.g. average vibration during the transport. The yellow is less likely to occur, such as a very low or high vibration. Similar to ASTM D4169-16 guidelines, corresponding assurance levels were plotted in solid black.

### Assurance Levels:

Level I: 99% of occurred vibrations beyond that line

Level II: 95% of occurred vibrations beyond that line

Level III: 90% of occurred vibrations beyond that line

For comparison the relevant ASTM D4169-16 guideline profiles were plotted as dotted blue lines.

The vibrations between the legs were clearly different. The air transport had the lowest vibration intensity, and the solid black PSD assurance levels were clearly below the blue dotted lines. Therefore, during this transport less intense stress was detected than typically tested during lab-based ASTM D4169-16 testing. A reason for this could be a smooth flight, absorption of vibrations by the thermal shipping system, or general improved aviation technology and a large airplane.

During truck transport, overall intensity was stronger than during air transport. Some frequencies between 8 to 20 Hz and below 3 Hz were more intense compared to general testing guidelines. Generally the measured vibrations are for most frequencies within or below expectations. The results are presented in FIG. 16.

### Transportation Legs and Modes

To compare the different legs of the transport, the data of the Reference loggers was compared. A similar amplification pattern was found for all transport routes. Frequencies between 10 and 60 Hz were amplified throughout the stack of packaging and frequencies above 200 Hz were damped. The results are in-line with lab scale testing where the same effect was observed.

### Shocks

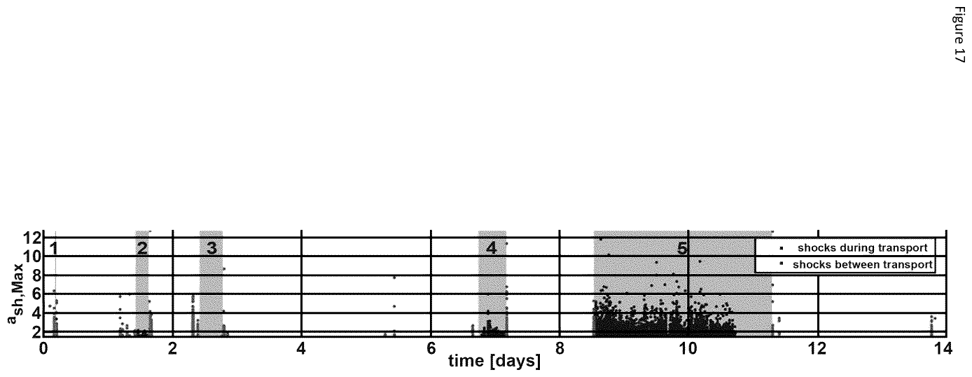

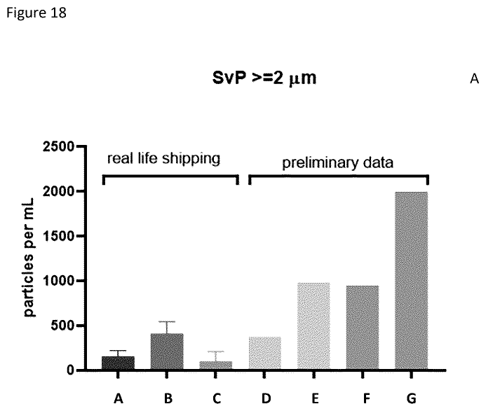

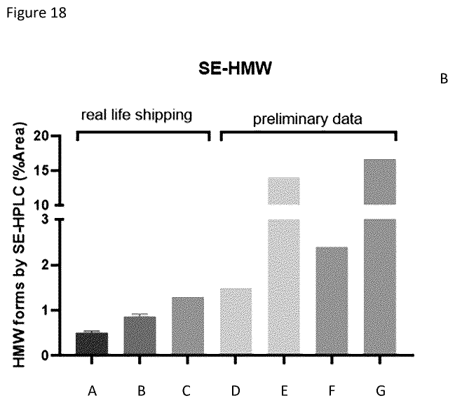

Most of the shocks were recorded during loading and unloading of the cargo, and during truck transport (leg 1, 2, 4 and 5) (FIG. 17).

### Product Quality

No impact on product stability was detected. The stress conditions did not result in measurable degradation with the selected model drugs. Subsequently, also no impact of the position of the drug product container within the pallet was detected.

No impact of the stress condition on the following quality attributes was found: Size variants, charge variants, colour, turbidity, visible particles, sub-visible particles.

### DISCUSSION

### Vibrations

Comprehensive set of vibration data shows the real-life stress conditions during transportation. Novel data on lateral vibrations was collected, and the data can serve for testing of vibrations in multiple axis. The overall energy input was lower than with current lab testing procedures. An effect of the position within the pallet was observed. PSD frequencies between 10-60 Hz are highest on top of the pallet and are higher than according to current lab testing guidelines. Therefore, a worst-case profile combining the highest PSD across the pallet can serve as future worst case testing profile.

### Product Quality Impact

No significant degradation or impact on quality attributes was detected. Neither the stable reference, nor any of the less stable formulations showed any degradation. The addition even of a small amount of surfactant (0.01% w/v) PS20 apparently protected the proteins from aggregating (FIG. 20). The vibration intensity and stress condition were therefore less intense than typically tested during development of drug products. The transport is assumed to be less intense than expected, and the drug product was stable enough to withstand these conditions.

During development, data on SvP formation and aggregate formation was observed with a shaking model system. The real-life transport is therefore less intense.

### EMBODIMENTS

In the following, further particular embodiments of the present invention are listed.

1. In an embodiment, a method for simulation of quality impact of transportation on a liquid is disclosed, said method comprising the steps of:
a) selecting a set of instructions comprising i) one or more PSD profile(s) comprising a multiplicity of frequencies and their corresponding amplitudes in at least two perpendicular axes (e.g. X and Z), and ii) a timetable specifying the duration for each of said PSD profiles(s);
b) inducing vibrations in the liquid according to said PSD profile(s); and
c) quality assessment and comparison of said liquid before and after performing the step b.

2. In an embodiment, the method of embodiment 1 is disclosed, wherein the vibrations in the two axes are induced simultaneously.

3. In an embodiment, the method of embodiment 1 or 2 is disclosed, wherein the liquid comprises a pharmaceutical product.

4. In an embodiment, the method of any of embodiments 1-3 is disclosed, wherein the liquid comprises a biological product.

5. In an embodiment, the method of embodiment 4 is disclosed, wherein the biological product is a protein (e.g. an antibody), nucleic acid, sugar, or conjugates and combinations thereof.

6. In an embodiment, the method of any of the preceding embodiments is disclosed, wherein the set of instructions as in embodiment 1a are different for different axes.

7. In an embodiment, the method of any of the preceding embodiments is disclosed, wherein the amplitude of vibrations in the first axis are proportional to the amplitude of vibrations in the second axis.

8. In an embodiment, the method of any of the preceding embodiments is disclosed, wherein the PSD profile is designed based on measurement of vibrations in one or more real life shipments.

9. In an embodiment, the method of any of the embodiments 1-8 is disclosed, wherein the frequency range in the PSD profile is between 1-300 Hz.

10. In an embodiment, the method of any of the embodiments 1-8 is disclosed, wherein the frequency range in the PSD profile is between 15-80 Hz.

11. In an embodiment, the method of any of the embodiments 1-7 is disclosed, wherein the PSD profile is as presented in FIG. 16 (A-G).

12. In an embodiment, the method of any of the embodiments 1-7 is disclosed, wherein the PSD profile is according to ASTM D4169 standards, e.g. Air level I, II, or III, Rail level I, II, or III, or Truck level I, II, or III.

13. In an embodiment, the method of any of the preceding embodiments is disclosed, wherein the quality assessment as in embodiment 1c is performed using analytical methods, e.g. Size-Exclusion Chromatography (SEC), Ion-Exchange Chromatography (IEC), Analytical ultra-centrifugation, visible or sub-visible particle analysis.

14. In an embodiment, a two-dimensional shaker adapted to execute the steps of the method of any of the preceding embodiments is disclosed.

15. In an embodiment, the two-dimensional shaker of embodiment 14 is disclosed, wherein the shaker comprises a temperature controlled chamber.

16. In an embodiment, the two-dimensional shaker of embodiment 15 is disclosed, wherein the chamber temperature is kept at 2-60 °C, e.g. at 2-8 °C.

17. In an embodiment, the two-dimensional shaker of any of embodiments 14-16 is disclosed, wherein the shaker comprises a vial holder and/or holder(s) for syringes.

18. In an embodiment, the two-dimensional shaker of embodiment 17 is disclosed, wherein the holder is adapted to function with different tilt angles.

19. In an embodiment, an apparatus for inducing vibrations simultaneously in at least two perpendicular directions (e.g. X and Z axis) in a liquid is disclosed.

20. In an embodiment, the apparatus of embodiment 19 is disclosed, wherein the apparatus comprises at least one separately controlled linear motor for each axis.

21. In an embodiment, the apparatus of embodiment 19 or 20 is disclosed, wherein the apparatus induces vibrations based on a set of instructions received by the apparatus comprising i) one or more PSD profile(s) comprising a multiplicity of frequencies and their corresponding amplitudes in at least two perpendicular axes (e.g. X and Z), and ii) a timetable specifying the duration for each of said PSD profile(s).

22. In an embodiment, the apparatus of any of embodiments 19-21 is disclosed, wherein the liquid comprises a pharmaceutical product.

23. In an embodiment, the apparatus of any of embodiments 19-22 is disclosed, wherein the liquid comprises a biological product.

24. In an embodiment, the apparatus of embodiment 23 is disclosed, wherein the biological product is a protein (e.g. an antibody), nucleic acid, sugar, or conjugates and combinations thereof.

25. In an embodiment, the apparatus of any of embodiments 19-24 is disclosed, wherein the set of instructions as in embodiment 21a are different for different axes.

26. In an embodiment, the apparatus of any of embodiments 19-25 is disclosed, wherein the PSD profile is designed based on measurement of vibrations in one or more real life shipments.

27. In an embodiment, the apparatus of any of the embodiments 19-26 is disclosed, wherein the frequency range in the PSD profile is between 1-300 Hz.

28. In an embodiment, the apparatus of any of the embodiments 19-26 is disclosed, wherein the frequency range in the PSD profile is between 15-80 Hz.

29. In an embodiment, the apparatus of any of the embodiments 19-25 is disclosed, wherein the PSD profile, i.e. the frequencies and their corresponding amplitudes, is as presented by solid lines or dotted lines in FIG. 16 (A-G) or within a 9-dB range in amplitude thereto.

30. In an embodiment, the apparatus of any of the embodiments 19-25 is disclosed, wherein the PSD profile is according to ASTM D4169 standards, e.g. Air level I, II, or III, Rail level I, II, or III, or Truck level I, II, or III.

31. In an embodiment, the apparatus of any of the embodiments 19-30 is disclosed, wherein the apparatus comprises a temperature controlled chamber.

32. In an embodiment, the apparatus of embodiments 31 is disclosed, wherein the chamber temperature is kept at 2-60 °C, e.g. at 2-8 °C.

33. In an embodiment, the apparatus of any of the embodiments 19-32 is disclosed, wherein the apparatus comprises a vial holder and/or holder(s) for syringes.

34. In an embodiment, the apparatus of embodiments 33 is disclosed, wherein the holder is adapted to function with different tilt angles.

35. In an embodiment, the apparatus of any of the embodiments 19-34 is disclosed for use in the method of any of the embodiments 1-13.

1. A method for simulation of quality impact of transportation on a liquid, said method comprising the steps of:
a) selecting a set of instructions comprising i) one or more PSD (Power Spectral Density) profile(s) comprising a multiplicity of frequencies and their corresponding amplitudes in at least two perpendicular axes (e.g. X and Z), and ii) a timetable specifying the duration for each of said PSD profiles(s);
b) inducing vibrations in the liquid according to said PSD profile(s); and
c) quality assessment and comparison of said liquid before and after performing the step b.

2. The method of claim 1, wherein the vibrations in the two axes are induced simultaneously.

3. The method of claim 1 or 2, wherein the liquid comprises a pharmaceutical product.

4. The method of any of claims 1-3, wherein the liquid comprises a biological product, such as a protein (e.g. an antibody), nucleic acid, sugar, or conjugates and combinations thereof.

5. The method of any of claims 1-4, wherein the set of instructions as in claim 1a are different for different axes and/or wherein the amplitude of vibrations in the first axis are proportional to the amplitude of vibrations in the second axis.

6. The method of any of claims 1-5, wherein the PSD profile is designed based on measurement of vibrations in one or more real life shipments.

7. The method of any of claims 1-6, wherein the frequency range in the PSD profile is between 1-300 Hz or between 15-80 Hz.

8. The method of any of claims 1-5, wherein the PSD profile, i.e. the frequencies and their corresponding amplitudes, is as presented by solid lines or dotted lines in FIG. 16 (A-G) or within a 9-dB range in amplitude thereto.

9. The method of any of claims 1-5, wherein the PSD profile is according to ASTM D4169 standards, e.g. Air level I, II, or III, Rail level I, II, or III, or Truck level I, II, or III.

10. The method of any of claims 1-9, wherein the quality assessment as in claim 1c is performed using analytical methods, e.g. Size-Exclusion Chromatography (SEC), Ion-Exchange Chromatography (IEC), Analytical ultra-centrifugation, visible or sub-visible particle analysis.

11. A two-dimensional shaker adapted to execute the method step as in claim 1b or any of claims 2-9 dependent thereto.

12. An apparatus for inducing vibrations simultaneously in at least two perpendicular directions (e.g. X and Z axis) in a liquid, optionally wherein the apparatus induces vibrations based on a set of instructions received by the apparatus comprising i) one or more PSD profile(s) comprising a multiplicity of frequencies and their corresponding amplitudes in at least two perpendicular axes (e.g. X and Z), and ii) a timetable specifying the duration for each of said PSD profile(s).

13. The apparatus of claim 12, wherein the PSD profile is designed based on measurement of vibrations in one or more real life shipments (e.g. as described in claim 8), and/or the frequency range in the PSD profile is between 1-300 Hz (e.g. 15-80 Hz).

14. The two-dimensional shaker of claim 11, or the apparatus of claim 12 or 13, further comprising a temperature controlled chamber, optionally wherein the chamber temperature is kept at 2-60 °C (e.g. 2-8 °C).

15. The two-dimensional shaker of claim 11 or 14, or the apparatus of any of claims 12-14, further comprising a vial holder and/or holder(s) for syringes, optionally wherein the holder is adapted to function with different tilt angles.
